(19) United States
(12) Patent Application Publication
(10) Pub. No.: US 2022/0170076 A1
(43) Pub. Date: Jun. 2, 2022

(54) METHOD FOR MANUFACTURING DEVICE, DEVICE, AND KIT

(71) Applicants: Hirotaka Unno, Tokyo (JP); Satoshi Nakazawa, Tokyo (JP); Yuuki Yonekawa, Tokyo (JP); Michie Hashimoto, Tokyo (JP); Fasmac Co., Ltd., Atsugi-shi (JP)

(72) Inventors: Hirotaka UNNO, Kanagawa (JP); Satoshi Nakazawa, Kanagawa (JP); Yuuki Yonekawa, Kanagawa (JP); Michie Hashimoto, Tokyo (JP); Satoshi Futo, Ebina-shi (JP); Mari Onishi, Kashiwa-shi (JP); Riztyan, Atsugi-shi (JP)

(73) Assignees: Ricoh Company, Ltd., Tokyo (JP); Fasmac Co., Ltd., Atsugi-shi (JP)

(21) Appl. No.: 17/455,971

(22) Filed: Nov. 22, 2021

(30) Foreign Application Priority Data
Nov. 30, 2020 (JP) 2020-198798
Jun. 10, 2021 (JP) 2021-097383

(51) Int. Cl. C12Q 1/6806; G01N 21/64

(52) U.S. Cl. CPC C12Q 1/6806; G01N 21/6428; G01N 2201/06113; G01N 2021/6439; G01N 21/6402

(57) ABSTRACT

A method for manufacturing a device is provided, in which a known quantity of a reagent is reliably immobilized in a reaction field, which can be stored at room temperature, and with which the performance of a real-time PCR apparatus can be correctly evaluated. The method is a method for manufacturing a device having at least one well, in which a specific number of copies of a nucleic acid in the at least one well are immobilized in a reaction field. The method includes a nucleic acid extraction step of extracting the nucleic acid with an enzyme and a drying deactivation step of deactivating the enzyme by drying at 5° C. to 45° C.

### CROSS-REFERENCE TO RELATED APPLICATIONS

This application claims priority to Japanese Patent Application No. 2020-198798, filed on Nov. 30, 2020, and Japanese Patent Application No. 2021-097383, filed on Jun. 10, 2021, the content of each of which is hereby incorporated by reference in its entirety.

### REFERENCE TO A SEQUENCE LISTING

The present application is accompanied by an ASCII text file as a computer readable form containing the sequence listing titled. “003765US_SL_ST25.txt”, created on Nov. 18, 2021, with a file size of 1,610 bytes, which is incorporated by reference in its entirety.

### BACKGROUND OF THE INVENTION

### Field of the Invention

The present invention relates to a method for manufacturing a device, a device, and a kit.

### Description of Related Art

PCR or qPCR (quantitative PCR) is used for qualitative and quantitative evaluation of a nucleic acid. For example, by examining the presence of a specific gene, it is possible to identify a variety, evaluate a genetic disease, or evaluate the presence or absence of a virus. So far, the performance assurance of an apparatus and the quality control of a measurement system for guaranteeing results have been carried out through temperature measurement of the heating block of the apparatus and the management of the apparatus by a user.

In recent years, as PCR has also been used in denial tests for genetically modified crop/food (GMO) and denial tests for virus contamination in the field of regenerative medicine, for example, the reliability of test results is required, and it is necessary to guarantee that the measurement system itself has accuracy sufficient to withstand the denial test and that the accuracy is maintained.

For example, Patent Document 1 describes a device in which a nucleic acid in at least one well is defined in the specific copy number.

### SUMMARY OF THE INVENTION

However, in the conventional device, since a known number of DNAs are provided in the form of a solution, there is a problem in that the expiration date is short due to the deterioration of DNA. In addition, it is conceivable that DNA adheres to a place other than the reaction field due to the movement of the solution, for example, during transportation, and thus a desired performance is not exhibited.

To solve these problems, it is conceivable to use refrigerated storage (storage at −20° C. or lower) and refrigerated transportation; however, there is a problem in that it is necessary to maintain and manage the frozen state.

The present invention provides a method for manufacturing a device, in which a known quantity of a reagent is reliably immobilized in a reaction field, which can be stored at room temperature, and with which the performance of a real-time PCR apparatus can be correctly evaluated.

The method for manufacturing a device of the present invention is a method for manufacturing a device having at least one well, in which a specific number of copies of a nucleic acid in the at least one well are immobilized in a reaction field, the method including a nucleic acid extraction step of extracting the nucleic acid with an enzyme and a drying deactivation step of deactivating the enzyme by drying at 5° C. to 45° C.

According to the method for manufacturing a device of the present invention, it is possible to provide a method for manufacturing a device, in which a known quantity of a reagent is reliably immobilized in a reaction field, which can be stored at room temperature, and with which the performance of a real-time PCR apparatus can be correctly evaluated.

### BRIEF DESCRIPTION OF THE DRAWINGS

FIG. 1 is a graph showing one example of the relationship between the frequency of DNA-replicated cells and the fluorescence intensity.

FIG. 2 is a schematic view showing one example of a solenoid valve type ejection head.

FIG. 3 is a schematic view showing one example of a piezo type ejection head.

FIG. 4 is a schematic view showing a modified example of the piezo type ejection head in FIG. 3.

FIG. 5A is a schematic view showing one example of a voltage applied to a piezoelectric element.

FIG. 5B is a schematic view showing another example of a voltage applied to a piezoelectric element.

FIG. 6A is a schematic view showing one example of a state of a liquid droplet.

FIG. 6B is a schematic view showing one example of a state of a liquid droplet.

FIG. 6C is a schematic view showing one example of a state of a liquid droplet.

FIG. 7 is a schematic view showing one example of a dispensing device for sequentially causing liquid droplets to land in a well.

FIG. 8 is a schematic view showing one example of a liquid droplet-forming device.

FIG. 9 is a view showing a hardware block of means for controlling the liquid droplet-forming device of FIG. 8.

FIG. 10 is a view showing a functional block of means for controlling the liquid droplet-forming device of FIG. 8.

FIG. 11 is a flowchart showing one example of the operation of the liquid droplet-forming device.

FIG. 12 is a schematic view showing a modified example of the liquid droplet-forming device.

FIG. 13 is a schematic view showing another modified example of the liquid droplet-forming device.

FIG. 14A is a view showing a case where flying liquid droplets contain two fluorescent particles.

FIG. 14B is a view showing a case where flying liquid droplets contain two fluorescent particles.

FIG. 15 is a view showing the relationship between a brightness value Li in a case where particles do not overlap with each other and an actually measured brightness value Le.

FIG. 16 is a schematic view showing another modified example of the liquid droplet-forming device.

FIG. 17 is a schematic view showing another example of a liquid droplet-forming device.

FIG. 18 is a schematic view showing one example of a method for counting cells that have passed through a micro flow path.

FIG. 19 is a schematic view showing one example of a method for acquiring an image of the vicinity of a nozzle unit of an ejection head.

FIG. 20 is a graph showing the relationship between the probability P (>2) and the average cell number.

FIG. 21A is a perspective view showing one example of a device.

FIG. 21B is a cross-sectional view taken along the line b-b′ in the arrow direction in FIG. 21A.

FIG. 22 is a block diagram showing one example of a hardware configuration of a performance evaluation device.

FIG. 23 is a block diagram showing one example of a functional configuration of the performance evaluation device.

FIG. 24 is a flowchart showing one example of performance evaluation program processing.

FIG. 25A is a table showing the in-plane distribution of Cq values of a targeted amplification product in Experimental Example 1.

FIG. 25B is a table showing evaluation results of the amplification reaction in Experimental Example 1.

FIG. 26A is a graph showing the relationship between the heating temperature and Cq Ave in Experimental Example 1.

FIG. 26B is a graph showing the relationship between the heating temperature and Cq σ in Experimental Example 1.

FIG. 27 is a graph showing the relationship between the copy numbers scattered based on the Poisson distribution and the coefficient of variation CV.

FIG. 28A is a table showing theoretical copy numbers of amplification products of a control sample (a reference) and an insoluble carrier sample in Experimental Example 5.

FIG. 28B is a table showing the in-plane distribution of Cq values of the amplification products of the control sample and the insoluble carrier sample.

FIG. 29 is a calibration curve showing the relationship between theoretical copy number and the Cq value of the control sample (a reference) in Experimental Example 5.

### DETAILED DESCRIPTION OF THE INVENTION

Hereinafter, a method for manufacturing a device, a device, and a kit, according to one embodiments of the present invention (hereinafter, may be simply referred to as a “manufacturing method of the present embodiment”, a “device of the present embodiment”, and a “kit of the present embodiment”, respectively) will be described with reference to the specific embodiments and drawings, as necessary. Such embodiments and drawings are merely examples for facilitating the understanding of the present invention and do not limit the present invention. That is, the shapes, dimensions, arrangements, or the like of the members described below can be changed and improved without departing from the gist of the present invention, and the present invention includes equivalents thereof.

Further, in all the drawings, the same constitutional elements are designated by the same reference numeral, and the description will not be duplicated.

In the present specification, all technical and scientific terms used have the same meaning as those commonly understood by persons skilled in the art, unless defined otherwise. All patents, applications, and other publications and information referred to in the present specification are incorporated herein by reference in their entirety. In addition, in a case where there is a conflict between the publication referenced in the present specification and the description in the present specification, the description in the present specification will prevail.

<Method for Manufacturing Device>

The method for manufacturing a device of the present embodiment is a method for manufacturing a device having at least one well, in which a specific number of copies of a nucleic acid in the at least one well are immobilized in a reaction field, the method including a nucleic acid extraction step of extracting the nucleic acid with an enzyme and a drying deactivation step of deactivating the enzyme by drying at 5° C. to 45° C.

Since the method for manufacturing a device of the present embodiment is a method for manufacturing a device having at least one well, in which a specific number of copies of a nucleic acid in the at least one well are immobilized in a reaction field, the method including a nucleic acid extraction step of extracting the nucleic acid with an enzyme and a drying deactivation step of deactivating the enzyme by drying at 5° C. to 45° C., it is possible to manufacture a device in which the nucleic acid is reliably immobilized in the reaction field and can be stored at room temperature.

As will be described later in Examples, according to the method for manufacturing a device of the present embodiment, it is possible to provide a device that can be stored at room temperature and with which a real-time PCR apparatus can be correctly evaluated since a nucleic acid is reliably immobilized in a reaction field in a well.

The real-time PCR quantifies a nucleic acid based on the amplification rate by measuring the amplification by PCR overtime (in real time). Quantification is carried out using a fluorescent dye, and the quantification using a fluorescence dye includes mainly an intercalation method and a hybridization method.

In the intercalation method, a nucleic acid is amplified in the presence of an intercalator that is specifically inserted (intercalates) into double-stranded DNA to emit fluorescence. Examples of the intercalator include SYBR Green I (CAS number: 163795-75-3) or a derivative thereof. On the other hand, in the hybridization method, a method using a TaqMan (registered trade name) probe is the most common, and a probe in which a fluorescent substance and a quenching substance are bonded to an oligonucleotide complementary to the target nucleic acid sequence is used.

In the performance evaluation method for a real-time PCR apparatus using the device manufactured by the method for a manufacturing device of the present embodiment, first, a nucleic acid amplification reaction is carried out using the real-time PCR apparatus. Subsequently, the amplification reaction is evaluated. The evaluation of the amplification reaction is preferably carried out based on the Cq value. One of these values may be used alone for evaluation, or two or more of thereof may be used in combination for evaluation.

The Cq value is synonymous with the threshold cycle value (Ct value) and means the number of PCR cycles at which a certain amount of an amplification product is obtained. A small Cq value indicates a large amount of nucleic acid is obtained, and a large Cq value indicates a small amount of nucleic acid is obtained. In the performance evaluation method of the present embodiment, the scattering of the Cq values refers to the scattering between Cq values individually obtained in reaction spaces in a case where the amplification reaction is carried out in a plurality of reaction spaces under the same conditions. A small scattering of Cq values means that the performance of a real-time PCR apparatus is high.

In the method for manufacturing a device of the present embodiment, the device has at least one well, in which a specific number of copies of a nucleic acid in the at least one well are immobilized in a reaction field, and the copy number is preferably 1 copy or more and 200 copies or less. In the manufacturing method of the present embodiment, both a reaction field in which a nucleic acid of the copy number of 200 or less is immobilized and a reaction field in which a nucleic acid of the copy number of more than 200 is immobilized may be present.

As will be described later in Examples, the performance evaluation of the real-time PCR apparatus can be carried out with high accuracy since the amount of a non-specific amplification product tends to increase in a case where the copy number of a nucleic acid immobilized in the reaction field is 200 copies or less, for example 180 copies or less, for example 170 copies or less, for example 160 copies or less, for example 150 copies or less, for example 140 copies or less, for example 130 copies or less, for example, 120 copies or less, for example 110 copies or less, for example 100 copies or less, for example 90 copies or less, for example 80 copies or less, for example 70 copies or less, for example 60 copies or less, for example 50 copies or less, for example 40 copies or less, for example 30 copies or less, for example, 20 copies or less, for example, 10 copies or less, for example, 5 copies or less, or for example, 1 copy.

[Specific Copy Number]

In the present specification, the description that a specific number of copies of a nucleic acid are immobilized in a reaction field means that the number of nucleic acids immobilized in the reaction field per well is specified with a predetermined degree of accuracy or higher. That is, it can be said that the number of nucleic acids actually immobilized in the reaction field of the well is known. That is, the specific copy number in the present specification is more accurate and reliable as the numerical number than the copy number (an estimated value by calculation) as determined by conventional serial dilution and in particular, is a controlled value regardless of the Poisson distribution even in the fewer copy number range of 1,000 copies or fewer.

For the controlled value, in general, the coefficient of variation CV, which represents uncertainty, is preferably within the value of either CV<1/√x with respect to the average copy number x or CV≤20%.

In the performance evaluation method of the present embodiment, since the copy number of the nucleic acid immobilized in the reaction field per well is specified, the performance evaluation of the real-time PCR apparatus can be carried out more accurately than before.

Here, the “copy number” of the nucleic acid and the “number of molecules” of the nucleic acid may be associated with each other. Specifically, for example, in the case of a G1 phase yeast in which a base sequence of a nucleic acid is introduced into two places on the genome, in a case where the number of yeasts is 1, the number of nucleic acid molecules (the number of the identical chromosome) is 1, and the copy number of the nucleic acid is 2. In the present specification, the specific copy number of the nucleic acid may be referred to as the absolute number of the nucleic acid.

In a case where there are a plurality of wells containing nucleic acid, the description that the same copy number of the nucleic acid is contained in each well means that the scattering in the number of nucleic acids, which occurs in a case where wells are filled with a reagent for carrying out amplification reaction, is within the allowable range. Whether or not the scattering in the number of nucleic acids is within the allowable range can be determined based on the information on uncertainty described below.

Examples of the information on the specific copy number of the nucleic acid include the information on uncertainty, the information on the carrier described later, and the information on the nucleic acid.

ISO/IEC Guide 99: 2007 [International Metrology Term—Basic and General Concept and Related Term (VIM)] defines that the “uncertainty” is a parameter that characterizes the scattering of values that accompany a measurement result and can be reasonably linked to the measured quantity”.

Here, “a value that could reasonably be linked to a measured quantity” means candidates for a true value of the measured quantity. That is, the uncertainty means information on the scattering of measurement results, which is derived from operations involved in the manufacture of the measurement target, equipment, or the like. The greater the uncertainty, the greater the scattering to be expected in the measurement result. The uncertainty may be, for example, a standard deviation obtained from the measurement result, or a half value of the confidence level indicated as the range of values in which the true value is included with at least a predetermined probability.

The uncertainty can be calculated based on Guide to the Expression of Uncertainty in Measurement (GUM: ISO/IEC Guide 98-3); Japan Accreditation Board Note 10, Guideline for measurement uncertainty in test; or the like.

As a method for calculating the uncertainty, for example, two methods of a type A evaluation method using statistics of measurement values and the like, and a type B evaluation method using the information on uncertainty obtained from a calibration certificate, a manufacturer's specification, published information, or the like can be applied.

The uncertainties can be expressed at the same confidence level by converting all the uncertainties obtained from factors such as operation and measurement into standard uncertainties. The standard uncertainty indicates the scattering of the average values obtained from the measurement values.

In one example of the method for calculating the uncertainty, for example, factors that cause uncertainties are extracted and the uncertainty (the standard deviation) of each of the factors is calculated. Subsequently, the calculated uncertainty of each of the factors is synthesized by the sum-of-squares method to calculate a synthetic standard uncertainty. Since the sum-of-squares method is used in the calculation of the synthetic standard uncertainty, among the factors that cause uncertainties, a factor providing a sufficiently small uncertainty can be ignored.

As the information on uncertainty, the coefficient of variation of the reagent filled in the reaction space may be used. The coefficient of variation means the relative value of the scattering in the number of nucleic acids with which each reaction space is filled, where the scattering occurs, for example, in a case where the reaction space is filled with the nucleic acid. That is, the coefficient of variation means the filling accuracy of the number of nucleic acids with which the reaction space is filled. The coefficient of variation is a value obtained by dividing the standard deviation a by the average value x. Here, in a case where a value obtained by dividing the standard deviation a by the average copy number (the average filling copy number) x is the coefficient of variation CV, a relational Expression 1 shown below is obtained.

$$CV = \frac{σ}{x} \qquad Expression 1$$

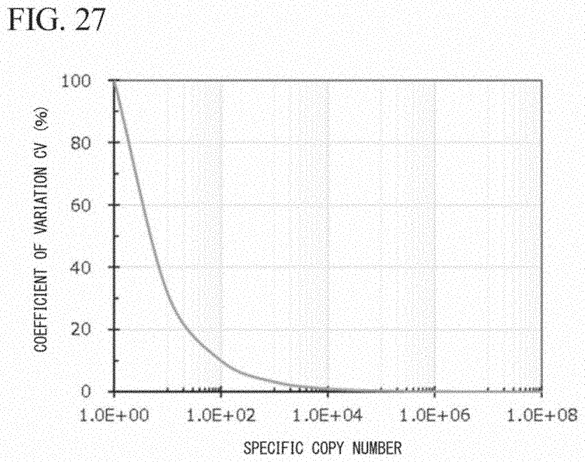

In general, nucleic acid is in a randomly distributed state of the Poisson distribution state in a dispersion solution. Therefore, in the serial dilution method, that is, in the random distribution state in the Poisson distribution, the standard deviation a and the average copy number x can be regarded to satisfy a relational Expression 2 shown below. From these, in a case where the dispersion solution of the nucleic acid is diluted by the serial dilution method and in a case where the coefficient of variation CV (the CV value) of the average copy number x is determined from the standard deviation a and the average copy number x by using Expression 3 shown below, which is derived from Expression 1 and Expression 2 shown above, the results are as shown in Table 1 and FIG. 27. The CV value of the coefficient of variation of the copy numbers having the scattering based on the Poisson distribution can be determined from FIG. 27.

$$\begin{matrix}σ = \sqrt{x} & Expression 2 \\ CV = \frac{1}{\sqrt{x}} & Expression 3\end{matrix}$$

TABLE 1

| Average copy number x | Coefficient of variation CV |
|---|---|
| 1.00E+00 | 100.00% |
| 1.00E+01 | 31.62% |
| 1.00E+02 | 10.00% |
| 1.00E+03 | 3.16% |
| 1.00E+04 | 1.00% |
| 1.00E+05 | 0.32% |
| 1.00E+06 | 0.10% |
| 1.00E+07 | 0.03% |
| 1.00E+08 | 0.01% |

From the results of Table 1 and FIG. 27, for example, in a case where the reaction space is filled with 100 copies of a nucleic acid by the serial dilution method, it can be found that the copy number of the nucleic acid with which the reaction space is filled finally has a coefficient of variation (a CV value) of at least 10% even in a case in which another accuracy is ignored.

Regarding the copy number of the nucleic acid, the CV value of the coefficient of variation and the average specific copy number x of the nucleic acid preferably satisfy the following expression, CV<1/√x, and more preferably satisfy CV<½√x.

As the information on uncertainty, in a case where there are a plurality of reaction spaces containing a nucleic acid, it is preferable to use the information on uncertainty of the entire reaction spaces as a whole, based on the specific copy number of the nucleic acid contained in the reaction spaces.

There are several possible factors that cause uncertainty, and examples thereof include, in a case where a nucleic acid is introduced into cells and the cells are counted and dispensed in the reaction space, the number of nucleic acids in a cell (for example, the change in the number of nucleic acids due to the cell cycle), means (including the result of the operation of each portion of the inkjet device, the device that controls the operation timing of the inkjet device, and the like, such as the number of cells contained in the liquid droplet in a case where the cell suspension is made into liquid droplets) for arranging cells in the reaction space, the frequency of cells being arranged in the appropriate position in the reaction space, (for example, the number of cells arranged in the reaction space) and the contamination due to mixing of the nucleic acid (contamination by a contaminant, which may be referred to as “contamination” hereinafter) in a cell suspension caused by cell destruction in the cell suspension.

Examples of the information on a nucleic acid include the information on the number of nucleic acids. Examples of the information regarding the number of nucleic acids include the information on uncertainty of the number of nucleic acids contained in the well.

The method for manufacturing a device of the present embodiment is a method for manufacturing a device having at least one well, in which a specific number of copies of a nucleic acid in the at least one well are immobilized in a reaction field, the method including a nucleic acid extraction step of extracting the nucleic acid with an enzyme and a drying deactivation step of deactivating the enzyme by drying at 5° C. to 45° C., preferably 10° C. to 45° C., more preferably 20° C. to 45° C., still more preferably 10° C. to 40° C., and particularly preferably 20° C. to 40° C. The drying deactivation method is not particularly limited as long as it can deactivate the enzyme in the above temperature range, and the drying may be drying under atmospheric pressure (1 atm) or may be drying under reduced pressure; however, drying under reduced pressure is preferable.

The reaction field is not particularly limited as long as it is a specific reaction space in the well; however, it is preferably the bottom surface of the well. In a case where the well is sealed with a sealing member, the reaction field may be a surface of an insoluble carrier serving as the sealing member, where the surface comes into contact with the reaction field. In addition, a nucleic acid may be directly immobilized on the reaction field; however, a nucleic acid may be indirectly immobilized on the reaction field by immobilizing the nucleic acid on an insoluble carrier and then adding the insoluble carrier on which the nucleic acid is immobilized to the reaction field.

The material of the insoluble carrier is not particularly limited as long as it is insoluble in the reaction solution, and can be appropriately selected depending on the intended purpose. Examples thereof include polystyrene, polypropylene, polyethylene, fluororesin, an acrylic resin, polycarbonate, polyurethane, polyvinyl chloride, polyethylene terephthalate, and a cyclic olefin copolymer (COC).

The method for manufacturing a device of the present embodiment includes a cell suspension generation step of generating a cell suspension containing a plurality of cells having a nucleic acid in a nucleus and a solvent, a liquid droplet-landing step of ejecting the cell suspension as liquid droplets to sequentially land the liquid droplets in the plate well, a cell number-measuring step of measuring the number of cells contained in the liquid droplet by a sensor after the ejection of the liquid droplet and before the landing of the liquid droplet in the well, a nucleic acid extraction step of extracting the nucleic acid with an enzyme from the cells in the well, and a drying deactivation step of deactivating the enzyme by drying at 5° C. to 45° C. It is more preferable to include a step of calculating the certainty of the number of nucleic acids estimated in the cell suspension generation step, the liquid droplet-landing step, and the cell number-measuring step, an output step, and a recording step. Further, other steps may be included as necessary.

(Cell Suspension Generation Step)

The cell suspension generation step is a step of generating a cell suspension containing a plurality of cells having a nucleic acid in a nucleus and a solvent. The solvent means a liquid that is used for dispersing cells. Regarding the cell suspension, the suspension means a solution in which cells are present in the state of being dispersed in a solvent. Generation means creating.

<<Cell Suspension>>

The cell suspension contains a plurality of cells having a nucleic acid in the nucleus, and a solvent, preferably contains an additive, and further contains other components as necessary. The plurality of cells having a nucleic acid in the nucleus are as described above.

<<Solvent>>

The solvent is not particularly limited, and can be appropriately selected depending on the intended purpose. Examples thereof include water, a culture solution, a separation solution, a diluent, a buffer solution, an organic substance solution, an organic solvent, a polymer gel solution, a colloidal dispersion solution, an aqueous electrolyte solution, an aqueous inorganic salt solution, an aqueous metal solution, and a mixed liquid thereof. One kind of these may be used alone, or two or more kinds thereof may be used in combination. Among these, water or a buffer is preferable, and water, phosphate-buffered saline (PBS), or a Tris-EDTA buffer (TE) is preferable.

<<Additive>>

The additive is not particularly limited, and can be appropriately selected depending on the intended purpose. Examples thereof include a surfactant, a nucleic acid, and a resin. One kind of these may be used alone, or two or more kinds thereof may be used in combination.

The surfactant can prevent the aggregation between cells and thus improve continuous ejection stability. The surfactant is not particularly limited, and can be appropriately selected depending on the intended purpose. Examples thereof include an ionic surfactant and a nonionic surfactant. One kind of these may be used alone, or two or more kinds thereof may be used in combination. Among these, a nonionic surfactant is preferable since a protein is not denatured and is deactivated, which depends on the amount added though.

Examples of the ionic surfactant include fatty acid sodium, fatty acid potassium, alpha sulfo fatty acid ester sodium, sodium linear alkylbenzene sulfonate, alkyl sulfate ester sodium, alkyl ether sulfate ester sodium, and sodium alpha olefin sulfonate. One kind of these may be used alone, or two or more kinds thereof may be used in combination. Among these, sodium fatty acid is preferable, and sodium dodecyl sulfate (SDS) is more preferable.

Examples of the nonionic surfactant include an alkyl glycoside, an alkyl polyoxyethylene ether (Brij series or the like), octylphenol ethoxylate (Triton X series, Igepal CA series, Nonidet P series, Nikkol OP series, or the like), polysorbates (Tween series such as Tween 20), sorbitan fatty acid ester, polyoxyethylene fatty acid ester, an alkyl maltoside, sucrose fatty acid ester, glycoside fatty acid ester, glycerin fatty acid ester, propylene glycol fatty acid ester, and fatty acid monoglyceride. One kind of these may be used alone, or two or more kinds thereof may be used in combination. Among these, polysorbates are preferable.

The content of the surfactant is not particularly limited, and can be appropriately selected depending on the intended purpose. However, the content of the surfactant is preferably 0.001% by mass or more and 30% by mass or less with respect to the total amount of the cell suspension. In a case where the content is 0.001% by mass or more, the effect of adding the surfactant can be obtained, and in a case where the content is 30% by mass or less, the aggregation of cells can be suppressed, and thus the copy number of the nucleic acid in the cell suspension can be strictly controlled.

The nucleic acid is not particularly limited as long as it does not affect the detection of the nucleic acid to be examined, and can be appropriately selected depending on the intended purpose. Examples thereof include ColE1 DNA. In a case where the nucleic acid is added, it is possible to prevent the nucleic acid from adhering to the wall surface or the like of the well.

The resin is not particularly limited, and can be appropriately selected depending on the intended purpose. Examples thereof include polyethyleneimide.

<<Other Components>>

Other components are not particularly limited, and can be appropriately selected depending on the intended purpose. Examples thereof include a cross-linking agent, a pH-adjusting agent, a preservative, an antioxidant, an osmotic pressure-adjusting agent, a wetting agent, and a dispersing agent.

The method for dispersing cells is not particularly limited, and can be appropriately selected depending on the intended purpose. Examples thereof include a media type method such as a bead mill, an ultrasonic type method such as an ultrasonic homogenizer, and a method using a pressure difference such as a French press. One kind of these may be used alone, or two or more kinds thereof may be used in combination. Among these, an ultrasonic type method is preferable since it causes less damage to cells. In the media type method, the cell membrane or cell wall may be destroyed, or the media may be mixed as a contaminant since the crushing ability is strong.

The cell screening method is not particularly limited, and can be appropriately selected depending on the intended purpose. Examples thereof include screening with wet type classification, a cell sorter, or a filter. One kind of these may be used alone, or two or more kinds thereof may be used in combination. Among these, screening with a cell sorter or a filter is preferable since the damage to cells is small.

Regarding the cells, it is preferable to estimate the number of nucleic acids from the number of cells contained in the cell suspension by measuring the cell cycle of the cell. Measuring the cell cycle means quantifying the number of cells affected by cell division. Estimating the number of nucleic acids means obtaining the copy number of the nucleic acid from the number of cells.

The counting target may not be the number of cells and may be the number of nucleic acids contained. Generally, the number of nucleic acids may be considered to be equal to the number of cells since cells into which one copy of the nucleic acid is introduced per one cell are selected or the nucleic acid is introduced into the cell by genetic recombination. However, since a cell undergoes cell division at a specific cycle, the replication of the nucleic acid occurs within the cell. The cell cycle differs depending on the kind of cell, but in a case of extracting a predetermined amount of solution from the cell suspension and measuring the cycles of a plurality of cells, the expected value for the number of nucleic acids contained in one cell and the certainty of the expected value can be calculated. This can be carried out, for example, by observing cells subjected to nuclear staining, with a flow cytometer.

The certainty means a probability of occurrence of a specific event, where the degree of possibility that the specific event will occur is predicted in advance in a case where several events are likely to occur. Calculation means to calculate and obtain a numerical value.

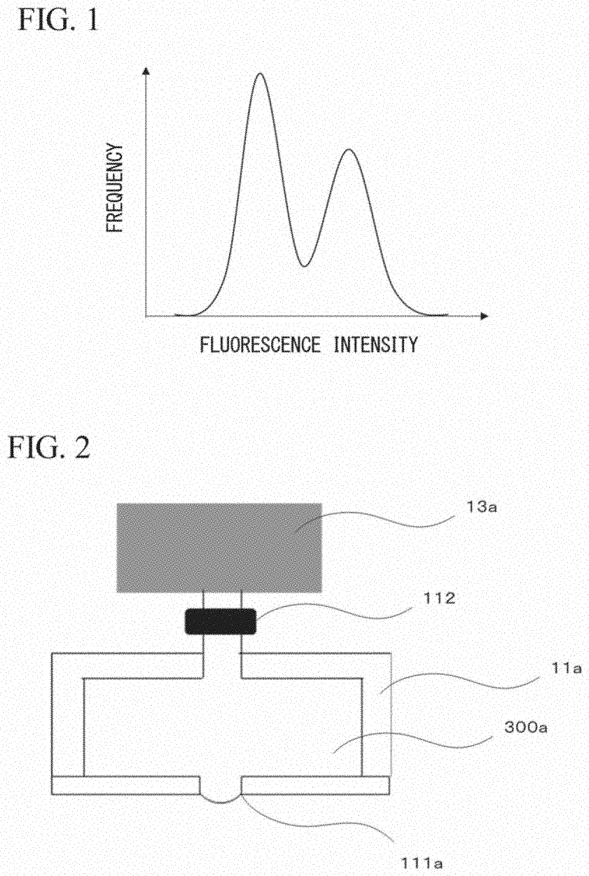

FIG. 1 is a graph showing one example of the relationship between the frequency of DNA-replicated cells and the fluorescence intensity. As shown in FIG. 1, since two peaks appear on the histogram depending on the presence or absence of DNA replication, it is possible to calculate the proportion of DNA-replicated cells. From this calculation result, it is possible to calculate the average number of nucleic acids contained in one cell, which is subsequently multiplied by the cell number measuring result described above, thereby the estimated number of nucleic acids being calculated.

In addition, it is preferable to perform a treatment for controlling the cell cycle before preparing a cell suspension. In a case where cells are synchronized in the state before or after the occurrence of the above-described replication, it is possible to more accurately calculate the number of nucleic acids from the number of cells.

It is preferable to calculate the certainty (the probability) of the specific copy number to be estimated. In a case where the certainty (probability) is calculated, it is possible to represent and output the certainty as a variance or a standard deviation based on these numerical values. In a case of summing up the effects of a plurality of factors, it is possible to use the square root of sum of squares of the standard deviations, which is commonly used. For example, the correct answer rate of the number of ejected cells, the number of DNA in the cell, the landing rate of the ejected cell that is made to land in the well, and the like can be used as factors. It is also possible to select, among these, an item that has a large influence and to perform a calculation.

(Liquid Droplet-Landing Step)

The liquid droplet-landing step is a step of ejecting the cell suspension as liquid droplets to sequentially land the liquid droplets in the plate well. The liquid droplet means a single mass of liquid that is formed by surface tension. “Ejecting” means causing a cell suspension to fly as liquid droplets. “Sequentially” means one after another in order. “Landing” means causing liquid droplets to reach the well.

As the ejecting means, means for ejecting a cell suspension as liquid droplets (hereinafter, may also be referred to as an “ejection head”) can be suitably used.

Examples of the method for ejecting a cell suspension as liquid droplets include an on-demand type method and a continuous type method in the inkjet method. Among these, in the case of the continuous type method, the dead volume of the cell suspension used tends to increase since empty ejection is continued until a stable ejection state is reached, the amount of liquid droplets is adjusted, and liquid droplets are continuously formed even in a case of moving between wells. In the present embodiment, it is preferable to reduce the influence of the dead volume from the viewpoint of adjusting the number of cells. For this reason, in the above two types of methods, the on-demand type method is more preferable.

Examples of the on-demand type method include a plurality of known methods such as a pressure application type method in which a liquid is ejected by applying pressure to the liquid, a thermal type method in which a liquid is ejected by film boiling due to heating, and an electrostatic type method in which liquid droplets are formed by pulling the liquid droplets by electrostatic attraction. Among these, a pressure application type method type is preferable for the following reasons.

In the electrostatic type method, it is necessary to install an electrode to face an ejection part that retains a cell suspension and forms liquid droplets. In the manufacturing method of the present embodiment, plates for receiving liquid droplets are disposed to face each other, and thus it is preferable that electrodes not be arranged in order to increase the degree of freedom in the plate configuration. In the thermal type method, local heating occurs, and thus there is a concern about the influence on cells, which are biological material, and scorching (cogation) on the heater part. Since the influence of heat depends on the content and the use of the plate, it is not necessary to exclude the influence of heat sweepingly, but the pressure application type method is preferable to the thermal type method since there is no concern about scorching on the heater unit.

Examples of the pressure application type method include a method for applying pressure to a liquid using a piezo element and a method for applying pressure by a valve such as a solenoid valve. Examples of the configuration of the liquid droplet generation device that can be used for the liquid droplet ejection of a cell suspension are shown in FIGS. 2 to 4.

FIG. 2 is a schematic view showing an example of a solenoid valve type ejection head. The solenoid valve type ejection head includes an electric motor 13a, a solenoid valve 112, a liquid chamber 11a, a cell suspension 300a, and a nozzle 111a. As the solenoid valve type ejection head, for example, a dispenser manufactured by Techelan LLC or the like can be preferably used.

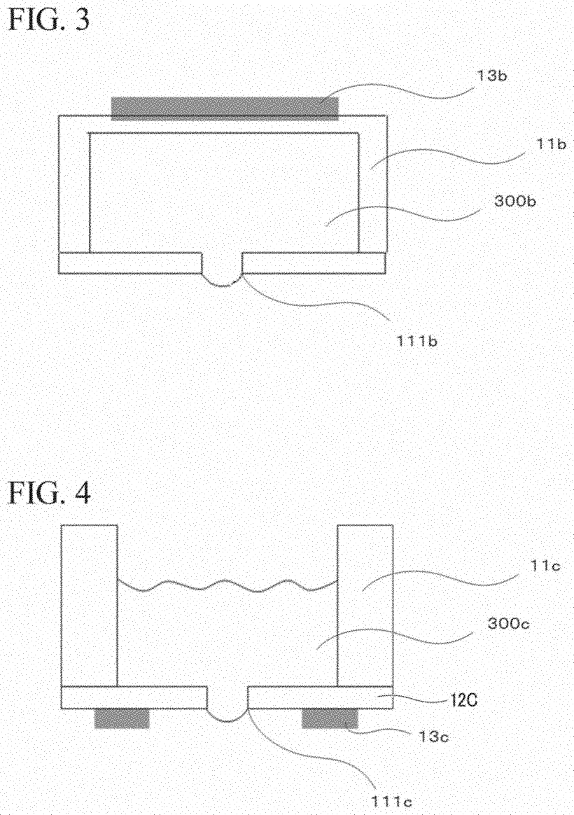

FIG. 3 is a schematic view showing an example of a piezo type ejection head. The piezo type ejection head has a piezoelectric element 13b, a liquid chamber 11b, a cell suspension 300b, and a nozzle 111b. As the piezo type ejection head, a single cell printer manufactured by Cytena Gmbh or the like can be preferably used.

Although any one of these ejection heads can be used, the piezo type method is preferably used to increase the throughput of plate formation since it is not possible to repeatedly form liquid droplets at high speed with the pressure application type method using a solenoid valve. Further, in the piezo type ejection head in which the general piezoelectric element 13b is used, non-uniformity of cell concentration due to sedimentation and nozzle clogging may occur as problems.

For this reason, as a more preferable configuration, a configuration shown in FIG. 4 or the like is mentioned. FIG. 4 is a schematic view showing a modified example of the piezo type ejection head in FIG. 3 in which a piezoelectric element is used. The ejection head of FIG. 4 has a piezoelectric element 13c, a liquid chamber 11c, a cell suspension 300c, and a nozzle 111c.

In the ejection head of FIG. 4, in a case where a voltage is applied to the piezoelectric element 13c from a control device which is not shown in the figure, compressive stress is applied in the lateral direction of the paper surface, and thus a membrane 12c can be deformed in the vertical direction of the paper surface.

Examples of the method other than the on-demand type method include a continuous type method in which liquid droplets are continuously formed. In the continuous type method, in a case where liquid droplets are pressurized and pushed out of the nozzle, a piezoelectric element or a heater provides a regular fluctuation, whereby fine liquid droplets can be continuously produced. Further, in a case where the ejection direction of the flying liquid droplet is controlled by applying a voltage, it is possible to select whether to land the liquid droplets in the well or collect the liquid droplets on the collection part. Such a method is used in a cell sorter or a flow cytometer, and for example, Cell Sorter SH800Z (apparatus name, manufactured by Sony Corporation) can be used.

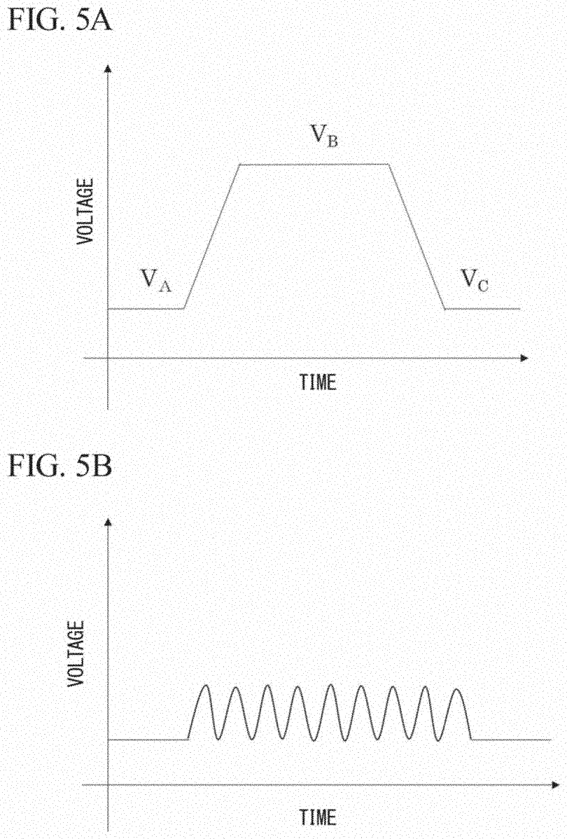

FIG. 5A is a schematic view showing one example of a voltage applied to a piezoelectric element. In addition, FIG. 5B is a schematic view showing another example of a voltage applied to a piezoelectric element. FIG. 5A shows a driving voltage for forming a liquid droplet. It is possible to form liquid droplets by controlling the value of voltage (V_{A}, V_{B}, V_{C}). FIG. 5B shows a voltage for stirring a cell suspension without ejecting a liquid droplet.

In a case of inputting a plurality of pulses that are not sufficient enough to eject a liquid droplet during the period in which a liquid droplet is not ejected, it is possible to stir a cell suspension in the liquid chamber, and thus the concentration distribution due to cell sedimentation can be suppressed.

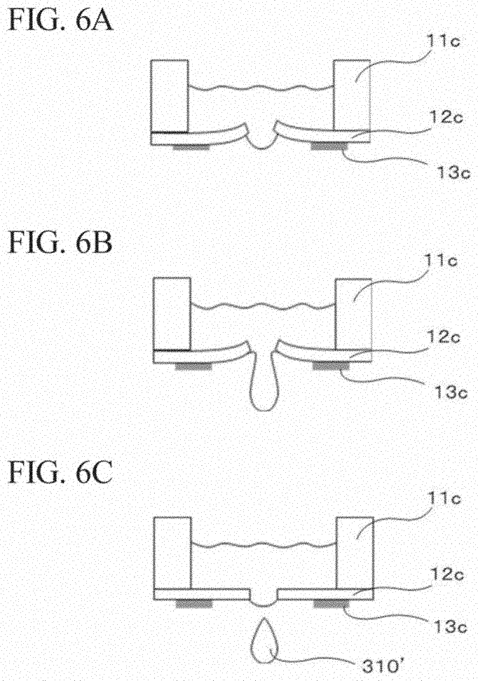

The liquid droplet-forming operation of the ejection head that can be used in the present embodiment will be described below. In a case where a voltage having a pulse form is applied to the upper and lower electrodes formed on the piezoelectric element, the ejection head can eject a liquid droplet. FIGS. 6 (a) to (c) are schematic views showing a state of a liquid droplet at each timing.

First, as shown in FIG. 6A, in a case of applying a voltage to the piezoelectric element 13c, the membrane 12c is rapidly deformed, whereby a high pressure is generated between the cell suspension retained in the liquid chamber 11c and the membrane 12c, and a liquid droplet is pushed out of the nozzle part by this pressure.

Next, as shown in FIG. 6B, the liquid is continuously ejected from the nozzle part until the pressure is reduced in the upper direction, and the liquid droplets grow. Finally, as shown in FIG. 6C, in a case where the membrane 12c returns to its original state, the liquid pressure in the vicinity of the interface between the cell suspension and the membrane 12c decreases, and a liquid droplet 310′ is formed.

In the manufacturing method of the present embodiment, the liquid droplets may be made to sequentially land in a well by fixing a plate, in which the well is formed, on a movable stage and combining the driving of the stage and the liquid droplets formation from the ejection head. Here, regarding the movement of the stage, the method for moving the plate has been described, but of course, the ejection head may be moved.

The plate is not particularly limited, and a plate in which a well (or wells) is formed, which is generally used in the biotechnology field, can be used. The number of wells in the plate is not particularly limited, and can be appropriately selected depending on the intended purpose. The plate may have one well or a plurality of wells.

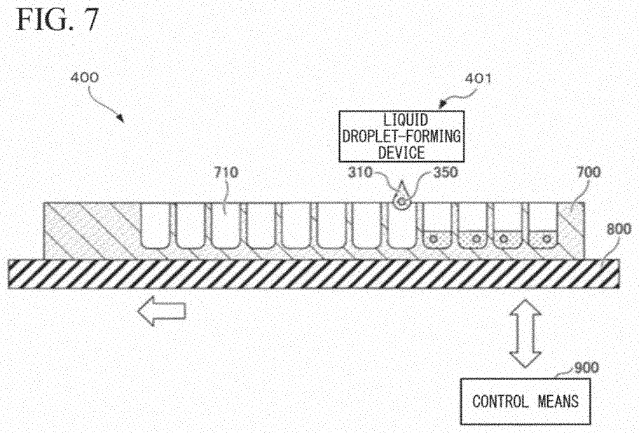

FIG. 7 is a schematic view showing one example of a dispensing device 400 for sequentially landing liquid droplets in a well of a plate. As shown in FIG. 7, the dispensing device 400 for landing liquid droplets includes a liquid droplet-forming device 401, a plate 700, a stage 800, and a control device 900.

In the dispensing device 400, the plate 700 is disposed on the stage 800, which is configured to be movable. In the plate 700, a plurality of wells (recessed parts) 710 on which the liquid droplet 310 is ejected from the ejection head of the liquid droplet-forming device 401 are provided. The control device 900 moves the stage 800 and controls the relative positional relationship between the ejection head of the liquid droplet-forming device 401 and each well 710. As a result, the liquid droplets 310 containing fluorescently stained cells 350 can be sequentially ejected from the ejection head of the liquid droplet-forming device 401 in each well 710.

The control device 900 can be configured to include, for example, a CPU, a ROM, a RAM, and a main memory. In this case, various functions of the control device 900 can be realized by reading out a program recorded in the ROM or the like into the main memory and executing the program by the CPU. However, a part or all of the control device 900 may be realized only by hardware. Further, the control device 900 may be physically composed of a plurality of devices and the like.

Regarding the liquid droplet to be ejected, it is preferable to cause the liquid droplet to land in the well so that a plurality of levels are obtained w % ben causing the cell suspension to land in the well. The plurality of levels means a plurality of standards that serve as standards. Examples of the plurality of levels include, for example, a predetermined concentration gradient of a plurality of cells, the nucleic acid of which is provided in the well. The plurality of levels can be controlled using the values measured by the sensor.

As the plate, it is preferable to use a 1-well microtube, an 8-well tube, a %-well plate, a 384-well plate, or the like. In a case where a plurality of wells are used, the same number of cells can be dispensed or different levels of the numbers of cells can be added in the wells of these plates. In addition, there may be wells containing no cells.

In a case where the well is sealed with an insoluble carrier serving as the sealing member, liquid droplets may be ejected onto the surface of the insoluble carrier serving as the sealing member, where the surface comes into contact with the reaction field. For example, liquid droplets may be ejected onto the back surface of the sealing member. In a case where the immobilization of the nucleic acid to the reaction field is the addition of the nucleic acid, which has been immobilized to the insoluble carrier, to the reaction field, liquid droplets are ejected to the insoluble carrier. In a case where the well is sealed with an insoluble carrier serving as the sealing member, the insoluble carrier serving as the sealing member may be used instead of the well in the above step, and in a case where the nucleic acid is immobilized to an insoluble carrier and the insoluble carrier is added to the reaction field, the insoluble carrier may be used instead of the well in the above step.

[Cell Number-Measuring Step]

The cell number-measuring step is a step of measuring the number of cells contained in the liquid droplet by a sensor after the liquid droplets are ejected and before the liquid droplets are made to land in the well. The sensor means a device that converts a mechanical, electromagnetic, thermal, acoustic, or chemical property of a natural phenomenon or artificial object, or spatial or temporal information indicated by the above property into a signal of another medium that is easily handled by a human or machine, by applying scientific principles. Measuring the number of cells means counting cells.

The cell number-measuring step is not particularly limited as long as the number of cells contained in the liquid droplet is measured by a sensor after the liquid droplet is ejected and before the liquid droplet is made to land in the well, and can be appropriately selected depending on the intended purpose. The cell number-measuring step may include a treatment for observing cells before ejection and a treatment for counting cells after landing.

For measuring the number of cells contained in the liquid droplet after the liquid droplet is ejected and before the liquid droplet is made to land in the well, it is preferable to observe the cells in the liquid droplet at the timing at which the liquid droplet is present directly above the well opening portion where the liquid droplet is predicted to reliably enter the well of the plate.

Examples of the method for observing the cells in the liquid droplet include a method for optically detecting and a method for electrically or magnetically detecting.

In a case where the well is sealed with an insoluble carrier serving as the sealing member, the insoluble carrier serving as the sealing member may be used instead of the well in the above step, and in a case where the nucleic acid is immobilized to an insoluble carrier and the insoluble carrier is added to the reaction field, the insoluble carrier is used instead of the well in the above step.

<<Method for Optically Detecting>>

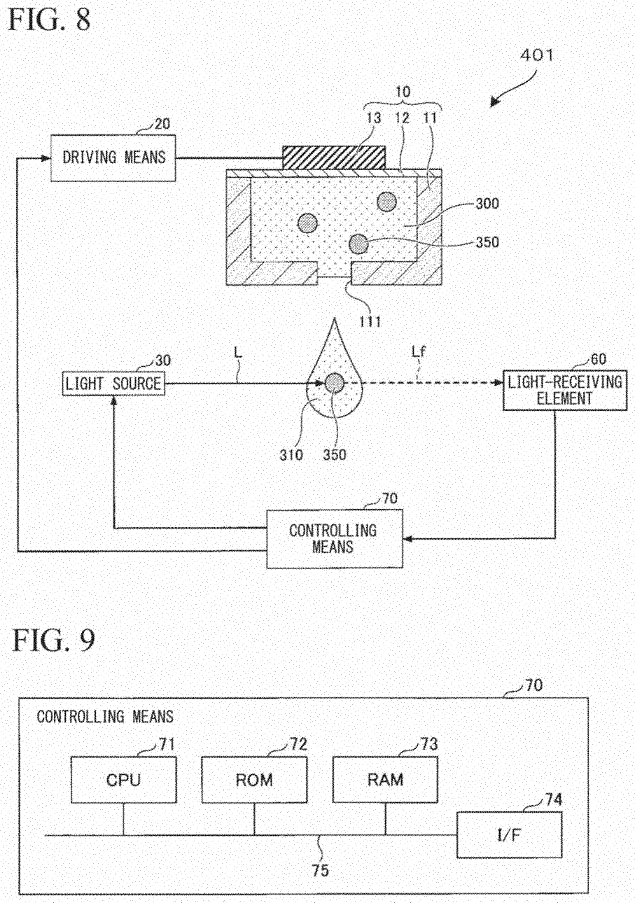

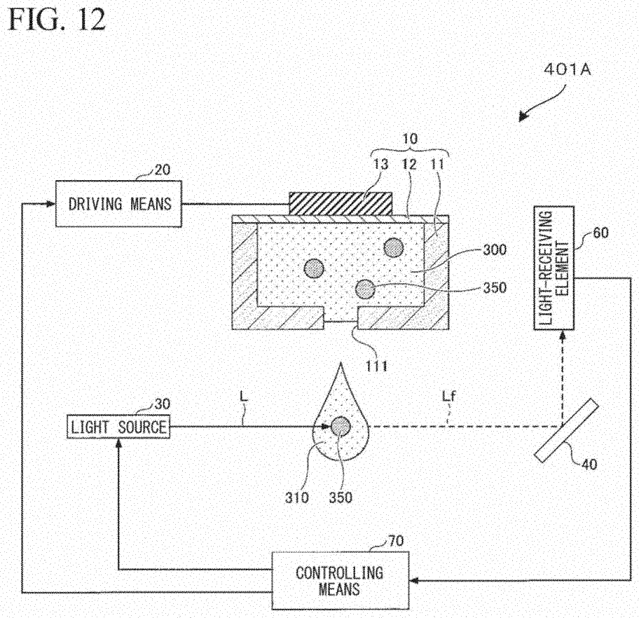

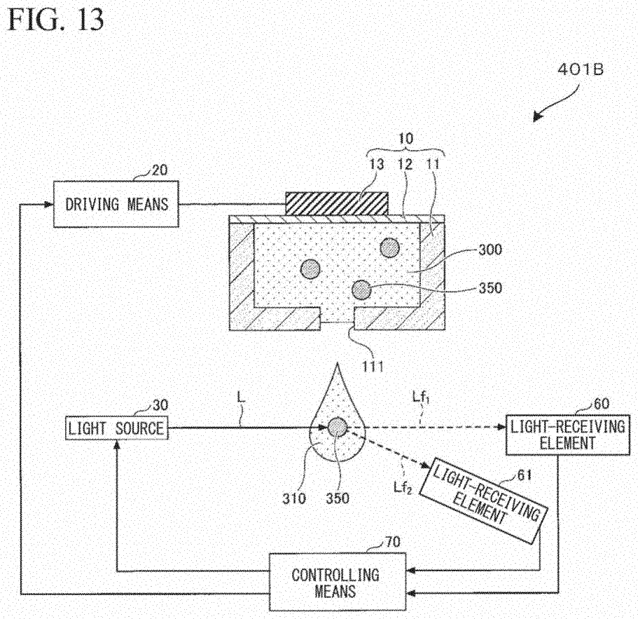

The method for optically detecting will be described below with reference to FIG. 8, FIG. 12, and FIG. 13. FIG. 8 is a schematic view showing one example of a liquid droplet-forming device 401. FIG. 12 and FIG. 13 are schematic views showing other examples (401A and 401B) of a liquid droplet-forming device. As shown in FIG. 8, the liquid droplet-forming device 401 includes an ejection head (a liquid droplet ejecting means) 10, a driving means 20, a light source 30, a light-receiving element 60, and a control means 70.

In FIG. 8, a liquid in which cells are fluorescently stained with a specific dye and then dispersed in a predetermined solution is used as a cell suspension, a liquid droplets formed from an ejection head are irradiated with light having a specific wavelength, which is emitted from a light source, and the cells emit fluorescence that is detected by a light-receiving element, whereby the cells are counted. At this time, in addition to the method of staining cells with a fluorescent dye, autofluorescence emitted by a molecule originally contained in the cells may be used. Alternatively, a gene encoding a fluorescent protein (for example, green fluorescent protein (GFP)) may be introduced into cells in advance so that the cells emit fluorescence. Irradiating with light means shedding light.

An ejection head 10 has a liquid chamber 11, a membrane 12, and a driving element 13, and can eject, as the liquid droplet, a cell suspension 300 in which fluorescently stained cells 350 are suspended.

The liquid chamber 11 is a liquid-retaining part for retaining the cell suspension 300 in which the fluorescently stained cells 350 are suspended, and a nozzle 111 which is a through-hole is formed on the lower surface side. The liquid chamber 11 can be formed of, for example, metal, silicon, ceramic, or the like. Examples of the fluorescently stained cell 350 include an inorganic fine particle and an organic polymer particle stained with a fluorescent dye.

The membrane 12 is a film-like member fixed at the upper end part of the liquid chamber 11. The plane shape of the membrane 12 can be, for example, circular, but may be elliptical, quadrangular, or the like.

The driving element 13 is provided on the upper surface side of the membrane 12. The shape of the driving element 13 can be designed in accordance with the shape of the membrane 12. For example, in a case where the plane shape of the membrane 12 is circular, it is preferable to provide the driving element 13 having a circular shape.

The membrane 12 can be vibrated by supplying a driving signal from the driving means 20 to the driving element 13. In a case where the membrane 12 is vibrated, the liquid droplet 310 containing the fluorescently stained cells 350 can be ejected from the nozzle 111.

In a case where a piezoelectric element is used as the driving element 13, for example, a structure can be provided in which electrodes for applying a voltage are provided on the upper surface and the lower surface of the piezoelectric material. In this case, in a case where a voltage is applied between the upper and lower electrodes of the piezoelectric element from the driving means 20, compressive stress is applied in the lateral direction of the paper surface, and thus a membrane 12 can be vibrated in the vertical direction of the paper surface. As the piezoelectric material, for example, lead zirconate titanate (PZT) can be used. In addition to the above, various piezoelectric materials such as bismuth iron oxide, metal niobate, barium titanate, and a material obtained by adding a metal or another oxide to these materials can be used.

The light source 30 irradiates a flying liquid droplet 310 with light L. The term “flying” means a state after the liquid droplet 310 is ejected from the liquid droplet ejecting means 10 and until it lands on the landing target object. The flying liquid droplet 310 is substantially spherical at the position where it is irradiated with the light L. In addition, the beam shape of the light L is substantially circular.

Here, it is preferable that the beam diameter of the light L be about 10 to 100 times the diameter of the liquid droplet 310. This is because the light source 30 reliably irradiates the liquid droplet 310 with the light L even in a case where the positions of the liquid droplets 310 are scattered.

However, it is preferable that the beam diameter of the light L not greatly exceed 100 times the diameter of the liquid droplet 310. This is because the energy density of the light with which the liquid droplet 310 is irradiated is decreased, the light quantity of fluorescence Lf emitted by using the light L as excitation light is decreased, and thus it is difficult for the light-receiving element 60 to detect the light.

The light L emitted from the light source 30 is preferably pulse light, and for example, a solid-state laser, a semiconductor laser, a dye laser, or the like is preferably used. In a case where the light L is pulse light, the pulse width is preferably 10 μs or less and more preferably 1 μs or less. The energy per unit pulse largely depends on the presence or absence of light collection or the like and the optical system but is generally preferably 0.1 μJ or more and more preferably 1 μJ or more.

In a case where the flying liquid droplet 310 contains the fluorescently stained cells 350, the light-receiving element 60 receives the fluorescence Lf emitted by the fluorescently stained cell 350 which absorbs the light L as excitation light. Since the fluorescence Lf is emitted from the fluorescently stained cell 350 in all directions, the light-receiving element 60 can be disposed at any position where the fluorescence Lf can be received. In this case, in order to improve the contrast, it is preferable to dispose the light-receiving element 60 at a position where the light L emitted from the light source 30 is not directly incident.

The light-receiving element 60 is not particularly limited as long as it is an element capable of receiving fluorescence Lf emitted from the fluorescently stained cell 350, and can be appropriately selected depending on the intended purpose; however, it is preferably an optical sensor that receives fluorescence from cells in the liquid droplet, which is emitted by irradiating the liquid droplet with light having a specific wavelength. Examples of the light-receiving element 60 include one-dimensional elements such as a photodiode and a photosensor, but it is preferable to use a photomultiplier tube or an avalanche photodiode in a case where a highly sensitive measurement is required. As the light-receiving element 60, for example, a two-dimensional element such as a charge-coupled device (CCD), a complementary metal-oxide-semiconductor (CMOS), or a gate CCD may be used.

Since the fluorescence Lf emitted by the fluorescently stained cell 350 is weaker than the light L emitted by the light source 30, a filter for damping the wavelength range of the light L may be installed in the front stage (the light-receiving surface side) of the light-receiving element 60. As a result, in the light-receiving element 60, an image of the fluorescently stained cell 350 having a very high contrast can be obtained. As the filter, for example, a notch filter that damps a specific wavelength range including the wavelength of light L can be used.

Further, as described above, the light L emitted from the light source 30 is preferably pulse light, but the light L emitted from the light source 30 may be continuously oscillating light. In this case, it is preferable to control the light-receiving element 60 so that the light-receiving element 60 is capable of incorporating light at the timing at which the flying liquid droplet 310 is irradiated with the continuously oscillating light and cause the light-receiving element 60 to receive the fluorescence Lf.

The controlling means 70 has a function of controlling the driving means 20 and the light source 30. Further, the control means 70 has a function of obtaining information based on the light quantity received by the light-receiving element 60 and measuring the number of fluorescently stained cells 350 (the case where the number is zero is included) contained in the liquid droplet 310. Hereinafter, the operation of the liquid droplet-forming device 401 including the operation of the controlling means 70 will be described with reference to FIG. 9 to FIG. 11.

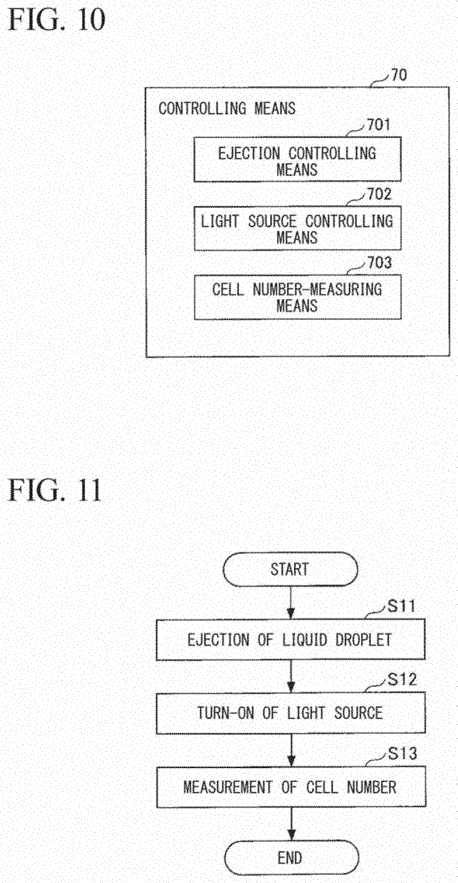

FIG. 9 is a view showing a hardware block of the controlling means of the liquid droplet-forming device of FIG. 8. FIG. 10 is a view showing a functional block of means for controlling the liquid droplet-forming device of FIG. 8. FIG. 11 is a flowchart showing one example of the operation of the liquid droplet-forming device.

As shown in FIG. 9, the controlling means 70 includes a CPU 71, a ROM 72, a RAM 73, a communication interface (a communication I/F) 74, and a bus line 75. The CPU 71, the ROM 72, the RAM 73, and the I/F 74 are connected to each other via a bus line 75.

The CPU 71 controls each of the functions of the controlling means 70. The ROM 72, which is a storing means, stores a program that is executed by the CPU 71 to control each of the functions of the controlling means 70 and various information. The RAM 73, which is a storing means, is used as a working area or the like of the CPU 71. In addition, the RAM 73 can temporarily store predetermined information. The I/F 74 is an interface for connecting the liquid droplet-forming device 401 to other devices or the like. The liquid droplet-forming device 401 may be connected to an external network or the like via the I/F 74.

As shown in FIG. 10, the controlling means 70 has, as a functional block, an ejection controlling means 701, a light source controlling means 702, and a cell number-measuring means (a cell number-detecting means) 703.

The cell number measuring of the liquid droplet-forming device 401 will be described with reference to FIG. 10 and FIG. 11. First, in a step S11, the ejection controlling means 701 of the controlling means 70 sends an ejection command to the driving means 20. The driving means 20 that receives the ejection command from the ejection controlling means 701 supplies a driving signal to the driving element 13 to vibrate the membrane 12. Due to the vibration of the membrane 12, the liquid droplet 310 containing the fluorescently stained cells 350 is ejected from the nozzle 111.

Next, in a step S12, the light source controlling means 702 of the controlling means 70 sends a lighting command to the light source 30 in synchronization with the ejection of the liquid droplet 310 (in synchronization with the driving signal supplied from the driving means 20 to the liquid droplet ejecting means 10). As a result, the light source 30 is turned on, and irradiates the flying liquid droplet 310 with the light L.

Here, the synchronization does not mean that the liquid droplet emits light at the same time as the liquid droplet 310 is ejected by the liquid droplet ejecting means 10 (at the same time that the driving means 20 supplies the driving signal to the liquid droplet ejecting means 10) but means that the light source 30 emits light at the timing at which the liquid droplet 310 is irradiated with the light L when the liquid droplet 310 flies and reaches a predetermined position. That is, the light source controlling means 702 controls the light source 30 so that the light is emitted with a delay of a predetermined time with respect to the ejection (the driving signal supplied from the driving means 20 to the liquid droplet ejecting means 10) of the liquid droplet 310 by the liquid droplet ejecting means 10.

For example, the velocity v of the liquid droplet 310 to be ejected when the driving signal is supplied to the liquid droplet ejection means 10 is measured in advance. Then, the time t required for reaching a predetermined position after the liquid droplet 310 is ejected is calculated based on the measured velocity v, and the light source 30 irradiates the light and the timing at which the light source emits light is delayed by t with respect to the timing at which the driving signal is supplied to the liquid droplet ejection means 10. As a result, good light emission control is possible, and the liquid droplet 310 can be reliably irradiated with the light from the light source 30.

Next, in a step S13, the cell number-measuring means 703 of the controlling means 70 measures the number of fluorescently stained cells 350 (the case where the number is zero is included) contained in the liquid droplet 310 based on the information from the light-receiving element 60. Here, the information from the light-receiving element 60 is a brightness value (a light quantity) or area value of the fluorescently stained cells 350.

The cell number-measuring means 703 can measure the number of fluorescently stained cells 350 by comparing, for example, the light quantity received by the light-receiving element 60 with a preset threshold value. In this case, a one-dimensional element or a two-dimensional element may be used as the light-receiving element 60.

In a case where a two-dimensional element is used as the light-receiving element 60, the cell number-measuring means 703 may perform the image processing technique for calculating the brightness value or area of the fluorescently stained cells 350 based on the two-dimensional image obtained from the light-receiving element 60. In this case, the cell number-measuring means 703 can calculate the number of fluorescently stained cells 350 by calculating the brightness values or area values of the fluorescently stained cells 350 by image processing or comparing the calculated brightness values or area values with a preset threshold value.

The fluorescently stained cell 350 may be a cell or a stained cell. The stained cell means a cell stained with a fluorescent dye or a cell capable of expressing a fluorescent protein. In the stained cells, the above-described fluorescent dye can be used. In addition, as the fluorescent protein, those described above can be used.

As described above, in the liquid droplet-forming device 401, the driving signal is supplied from the driving means 20 to the liquid droplet-ejecting means 10 retaining the cell suspension 300 in which the fluorescently stained cells 350 are suspended, the liquid droplet 310 containing the fluorescently stained cells 350 is ejected, and the flying liquid droplet 310 is irradiated with the light L from the light source 30. Then, the fluorescently stained cells 350 contained in the flying liquid droplet 310 emit fluorescence Lf using the light L as excitation light, and the light-receiving element 60 receives the fluorescence Lf. Further, based on the information from the light-receiving element 60, the cell number-measuring means 703 measures the number of (counts) fluorescently stained cells 350 contained in the flying liquid droplet 310.

That is, in the liquid droplet-forming device 401, since the number of fluorescently stained cells 350 contained in the flying liquid droplet 310 is actually observed on the spot, the measurement accuracy of the number of fluorescently stained cells 350 is improved as compared with the conventional case. Further, since the fluorescently stained cells 350 contained in the flying liquid droplets 310 are irradiated with the light L to emit the fluorescence Lf and then the fluorescence Lf is received by the light-receiving element 60, an image of the fluorescently stained cells 350 can be obtained with high contrast, whereby it is possible to reduce the frequency of occurrence of an erroneous measurement of the number of fluorescently stained cells 350.

FIG. 12 is a schematic view showing a modified example of the liquid droplet-forming device 401 of FIG. 8. As shown in FIG. 12, a liquid droplet-forming device 401A is different from the liquid droplet-forming device 401 (see FIG. 8) in that a mirror 40 is disposed in front of the light-receiving element 60. It is to be noted that the description of the same component as that of the embodiment described above may be omitted.

As described above, in the liquid droplet-forming device 401A, the degree of freedom in the layout of the light-receiving element 60 can be improved by disposing the mirror 40 in front of the light-receiving element 60.

For example, in a case where the nozzle 111 is brought to be close to the landing target object, interference may occur between the landing target object and the optical system (particularly, the light-receiving element 60) of the liquid droplet-forming device 401 in the layout of FIG. 8. However, in a case where the layout shown in FIG. 12 is adopted, it is possible to avoid the occurrence of interference.

In a case of changing the layout of the light-receiving element 60 as shown in FIG. 12, it is possible to reduce the distance (gap) between the lading target object on which the liquid droplet 310 lands and the nozzle 111, and thus the scattering of landing positions can be suppressed. As a result, it is possible to improve the accuracy of dispensing.

FIG. 13 is a schematic view showing another modified example of the liquid droplet-forming device 401 of FIG. 8. As shown in FIG. 13, the liquid droplet-forming device 401B is different from the liquid droplet-forming device 401 (see FIG. 8) in that a light-receiving element 61 that receives fluorescence Lf₂ emitted from the fluorescently stained cell 350 is provided in addition to the light-receiving element 60 that receives the fluorescence Lf₁ emitted from the fluorescently stained cell 350. It is to be noted that the description of the same component as that of the embodiment described above may be omitted.

Here, the fluorescences Lf₁ and Lf₂ indicate some of the fluorescence emitted from the fluorescently stained cell 350 in all directions. The light-receiving elements 60 and 61 can be disposed at any positions at which the fluorescence emitted from the fluorescently stained cell 350 in different directions can be received. It is to be noted that three or more light-receiving elements may be disposed at positions at which the fluorescence emitted from the fluorescently stained cell 350 in different directions can be received. Further, each of the light-receiving elements may have the same specification or may have different specifications from each other.

In a case where there is only one light-receiving element, there is a risk that the cell number-measuring means 703 will erroneously measure the number of (erroneously count) fluorescently stained cells 350 contained in the liquid droplet 310 due to the overlapping of the fluorescently stained cells 350, in a case where the flying liquid droplet 310 contains a plurality of fluorescently stained cells 350.

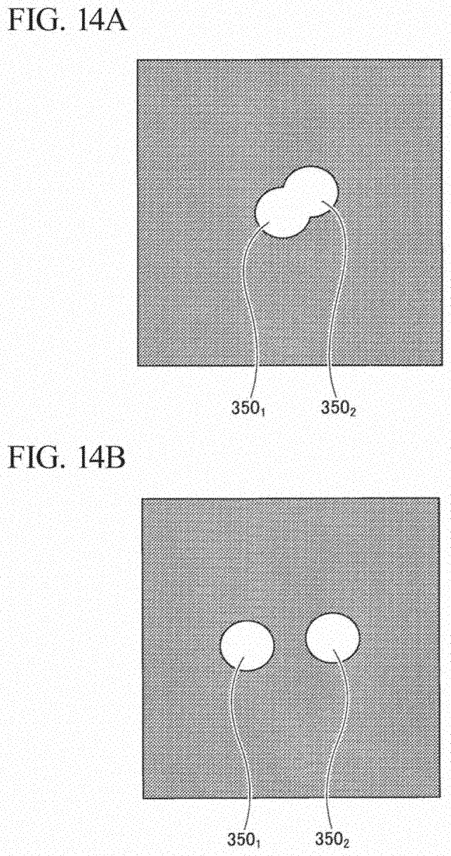

FIG. 14A and FIG. 14B are views showing a case where a flying liquid droplet contains two fluorescently stained cells. For example, there may be a case where the fluorescently stained cells 350₁ and 350₂ overlap, as shown in FIG. 14A, or a case where the fluorescently stained cells 350₁ and 350₂ do not overlap, as shown in FIG. 14B. In a case where two or more light-receiving elements are provided, it is possible to reduce the influence of overlapping of fluorescently stained cells.

As described above, the cell number-measuring means 703 can calculate the number of fluorescent particles by calculating the brightness values or area values of the fluorescent particles by image processing and comparing the calculated brightness values or area values with a preset threshold value.

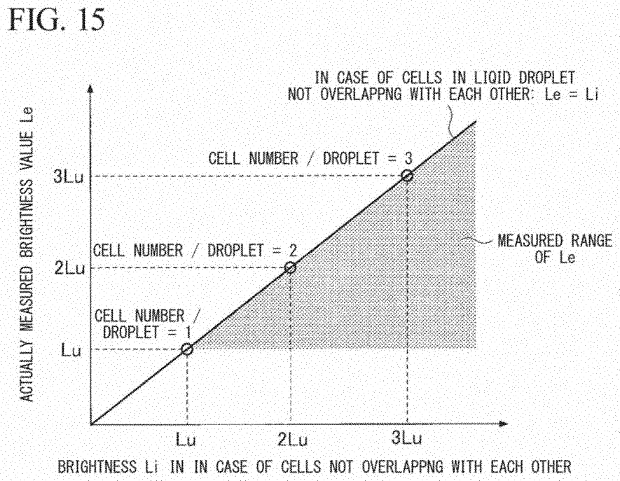

In a case where two or more light-receiving elements are installed, it is possible to suppress the occurrence of a counting error by adopting data indicating the maximum value among the brightness values or area values obtained from each of the light-receiving elements. This will be described in more detail with reference to FIG. 15.

FIG. 15 is a view showing the relationship between a brightness value Li in a case where particles do not overlap with each other and an actually measured brightness value Le. As shown in FIG. 15, in a case where there is no overlap between the particles in the liquid droplet, an expression Le=Li holds. For example, in a case where the brightness value of one cell is denoted by Lu, an expression Le=Lu holds in a case where the number of cells per drop is 1, and an expression Le=n Lu holds in a case where the number of cells per drop is n (n: natural number).

However, in reality, in a case where n is 2 or more, particles may overlap with each other, and thus the actually measured brightness value Le is Lu≤Le≤n Lu (corresponding to the shaded portion in FIG. 15). Accordingly, in a case where the number of cells per drop is n, the threshold value can be set as, for example, (n Lu−Lu/2)≤threshold value<(n Lu+Lu/2). In a case where a plurality of light-receiving elements are installed, it is possible to suppress the occurrence of a counting error by adopting data indicating the maximum value among the data obtained from each of the light-receiving elements. The area value may be used instead of the brightness value.

Further, in a case where a plurality of light-receiving elements are installed, the number of cells may be determined by an algorithm for estimating the number of cells based on the obtained plurality of shape data. As described above, since the liquid droplet-forming device 401B has a plurality of light-receiving elements that receive the fluorescence emitted from the fluorescently stained cells 350 in different directions, the frequency of occurrence of an erroneous measurement of the number of fluorescently stained cells 350 can be further reduced.

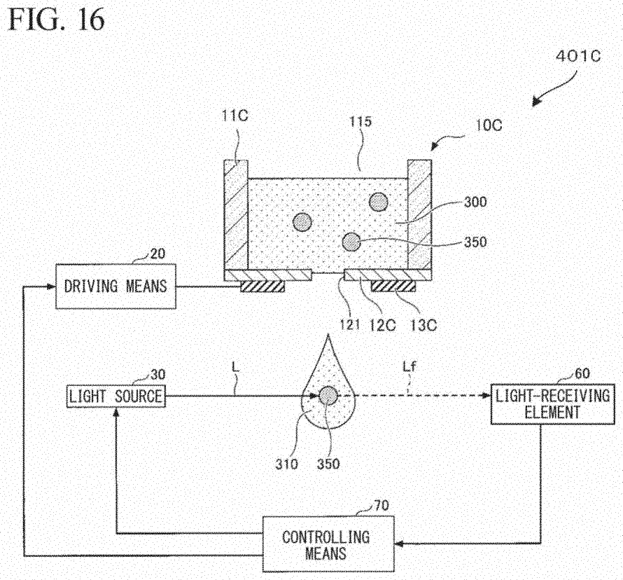

FIG. 16 is a schematic view showing another modified example of the liquid droplet-forming device 401 of FIG. 8. As shown in FIG. 16, a liquid droplet-forming device 401C is different from the liquid droplet-forming device 401 (see FIG. 8) in that the liquid droplet ejecting means 10 is replaced with a liquid droplet ejecting means 10C. It is to be noted that the description of the same component as that of the embodiment described above may be omitted.

The liquid droplet-ejecting means 10C has a liquid chamber 11C, a membrane 12C, and a driving element 13C. The liquid chamber 11C has, at the upper part thereof, an atmospheric air opening part 115 that opens the inside of the liquid chamber 11C to the atmosphere and is configured so that air bubbles mixed in the cell suspension 300 can be discharged from the atmospheric air opening part 115.

The membrane 12C is a film-like member fixed at the lower end part of the liquid chamber 11C. A nozzle 121, which is a through-hole, is formed at the substantial center of the membrane 12C, and the cell suspension 300 retained in the liquid chamber 11C is ejected as the liquid droplet 310 from the nozzle 121 by the vibration of the membrane 12C. Since the liquid droplet 310 is formed by the inertia of the vibration of the membrane 12C, even the cell suspension 300 having a high surface tension (a high viscosity) can be ejected. The plane shape of the membrane 12C can be, for example, circular, but may be elliptical, quadrangular, or the like.

The material of the membrane 12C is not particularly limited, but in a case where the material is too soft, the membrane 12C vibrates easily, and thus it is difficult to immediately suppress the vibration when performing ejection. Accordingly, it is preferable to use a material having a certain degree of hardness. As the material of the membrane 12C, for example, a metal material, a ceramic material, a polymer material having a certain degree of hardness, or the like can be used.

In particular, in a case where cells are used as fluorescently stained cells 350, it is preferable that the material have low attachability to a cell and a protein. It is generally said that the attachability of a cell depends on the contact angle of water on the material, and in a case where the material has low hydrophilicity or high hydrophobicity, the cell attachability is low. Various metal materials and ceramics (metal oxides) can be used as the material having high hydrophilicity, and a fluororesin or the like can be used as the material having high hydrophobicity.

Other examples of such materials include stainless steel, nickel, aluminum and the like, silicon dioxide, alumina, and zirconia. Apart from the above, it is also conceivable to reduce cell adhesiveness by coating the surface of the material. For example, the surface of the material can be coated with the above-described metal or metal oxide material, or with a synthetic phospholipid polymer mimicking a cell membrane (for example, Lipidure manufactured by NOF Corporation).

It is preferable that the nozzle 121 be formed as a substantially circular through-hole at the substantial center of the membrane 12C. In this case, the diameter of the nozzle 121 is not particularly limited, but it is preferably at least two times the size of the fluorescently stained cells 350 in order to prevent the nozzle 121 from being clogged by the fluorescently stained cells 350. In a case where the fluorescently stained cell 350 is, for example, an animal cell, particularly a human cell, since the size of the human cell is generally about 5 μm to 50 μm, the diameter of the nozzle 121 is preferably 10 μm or more and more preferably 100 μm or more in accordance with the cell to be used.

On the other hand, in a case where the liquid droplet is too large, it is difficult to achieve the purpose of forming a fine liquid droplet, and thus the diameter of the nozzle 121 is preferably 200 μm or less. That is, in the liquid droplet-ejecting means 10C, the diameter of the nozzle 121 is typically in the range of 10 μm to 200 μm.

The driving element 13C is formed on the lower surface side of the membrane 12C. The shape of the driving element 13C can be designed in accordance with the shape of the membrane 12C. For example, in a case where the plane shape of the membrane 12C is circular, it is preferable to form a driving element 13C having the plane shape of an annular shape (a ring shape) around the nozzle 121. The driving system of the driving element 13C can be the same as that of the driving element 13.

The driving means 20 can selectively (for example, alternately) apply an ejection wave form for vibrating the membrane 12C to form the liquid droplet 310 and a stirring wave form for vibrating the membrane 12C within a range that does not form the liquid droplet 310 to the driving element 13C.

For example, in a case of making both the ejection wave form and the stirring wave form a rectangular wave and lowering the driving voltage of the stirring wave form in comparison with the driving voltage of the ejection wave form, it is possible to prevent the liquid droplet 310 from being formed by applying the stirring wave form. That is, the vibration state (the degree of vibration) of the membrane 12C can be controlled by the level of the driving voltage.

In the liquid droplet-ejecting means 10C, since the driving element 13C is formed on the lower surface side of the membrane 12C, in a case where the membrane 12 vibrates due to the driving element 13C, a flow from the lower side to the upper side of the liquid chamber 11C can be generated.

In this case, the movement of the fluorescently stained cell 350 is a movement from the bottom side to the top side, and convection occurs in the liquid chamber 11C, whereby the cell suspension 300 containing the fluorescently stained cells 350 is stirred. Due to the flow from the lower side to the upper side of the liquid chamber 11C, the sedimented and aggregated fluorescently stained cells 350 are uniformly dispersed inside the liquid chamber 11C.

That is, the driving means 20 applies the ejection wave form to the driving element 13C and controls the vibration state of the membrane 12C, and thus the cell suspension 300 retained in the liquid chamber 11C can be ejected from the nozzle 121 as the liquid droplet 310. In addition, the driving means 20 applies the stirring wave form to the driving element 13C and controls the vibration state of the membrane 12C, and thus the cell suspension 300 retained in the liquid chamber 11C can be stirred. At the time of stirring, the liquid droplet 310 is not ejected from the nozzle 121.

In a case where the cell suspension 300 is stirred in this manner while the liquid droplets 310 are not formed, it is possible to prevent the fluorescently stained cells 350 from regimenting and aggregating on the membrane 12C and possible to uniformly disperse the fluorescently stained cells 350 in the cell suspension 300. As a result, it is possible to suppress the clogging of the nozzle 121 and the scattering of the numbers of fluorescently stained cells 350 in the ejected liquid droplet 310. As a result, the cell suspension 300 containing the fluorescently stained cells 350 can be continuously and stably ejected as liquid droplets 310 for a long period of time.

Further, in the liquid droplet-forming device 401C, air bubbles may be mixed in the cell suspension 300 in the liquid chamber 11C. Even in this case, in the liquid droplet-forming device 401C, since the atmospheric air opening part 115 is provided at the upper part of the liquid chamber 11C, the air bubbles mixed in the cell suspension 300 can be discharged to the outside atmospheric air through the atmospheric air opening part 115. As a result, it is possible to continuously and stably form the liquid droplets 310 without consuming a large amount of liquid for discharging air bubbles.

That is, in a case where air bubbles are mixed in the vicinity of the nozzle 121 or in a case where a large number of air bubbles are mixed in the membrane 12C, the ejection state is affected. Therefore, in order to stably form the liquid droplets for a long period of time, it is necessary to discharge the mixed air bubbles. Typically, the air bubbles mixed in the membrane 12C move upward naturally or by the vibration of the membrane 12C. However, since the atmospheric air opening part 115 is provided in the liquid chamber 11C, the mixed air bubbles can be discharged through the atmospheric air opening part 115. Therefore, even in a case where air bubbles are mixed in the liquid chamber 11C, it is possible to prevent non-ejection from occurring, and the liquid droplet 310 can be continuously and stably formed.

At the timing at which the liquid droplets are not formed, the membrane 12C may be vibrated within a range where the liquid droplets are not formed so that the air bubbles are actively moved to the upside of the liquid chamber 11C.

<<Method for Electrically or Magnetically Detecting>>

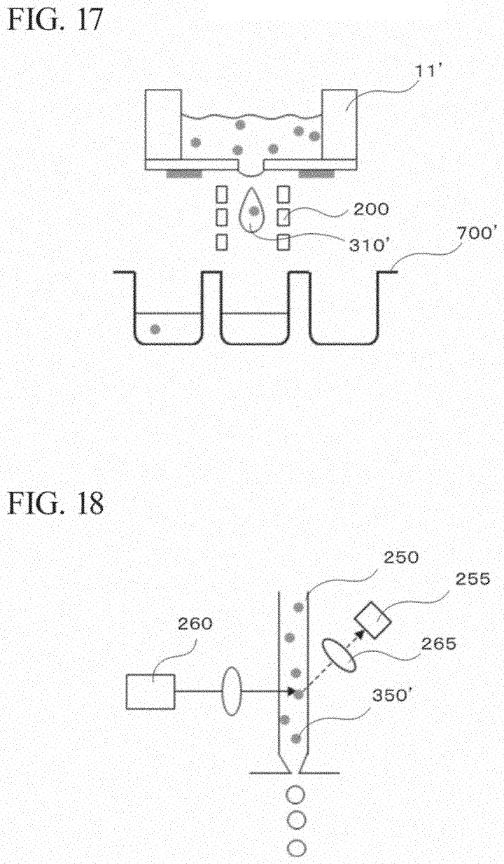

For the method for electrically or electrically detecting, as shown in FIG. 17, a coil 200 for measuring the number of cells is installed as a sensor directly under an ejection head that ejects a cell suspension as a liquid droplet 310′ from a liquid chamber 11′ to a plate 700′. In a case where cells are covered with magnetic beads that have been modified by a specific protein and capable of adhering to the cells, it is possible to detect the presence or absence of the cells in the flying liquid droplet due to the induced current that is generated as the cells to which magnetic beads are attached pass through the coil. Generally, a cell has a protein unique to the cell on the surface thereof, and thus it is possible to attach a magnetic bead to the cell by modifying the magnetic bead with an antibody capable of adhering to the protein. A ready-made product can be used as such magnetic beads, and for example, Dynabeads (registered trade mark) manufactured by VERITAS Corporation can be used.

<<Treatment for Observing Cells Before Ejection>>

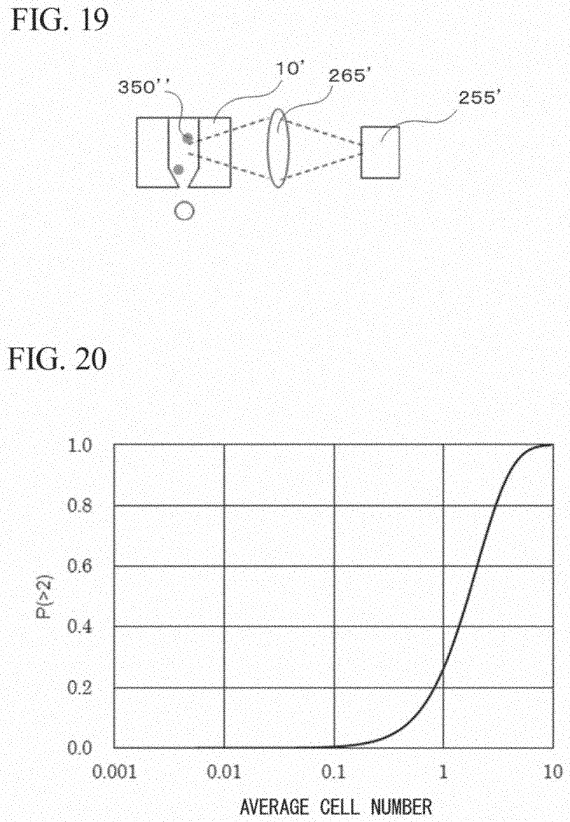

Examples of the treatment for observing cells before ejection include a method for counting cells 350′ which have passed through a micro flow path 250 shown in FIG. 18 and a method for acquiring an image of the vicinity of the nozzle part of the ejection head shown in FIG. 19.

The method shown in FIG. 18 is a method used in a cell sorter apparatus, and for example, a cell sorter SH800Z manufactured by Sony Corporation can be used. In FIG. 18, it is possible to form liquid droplets while identifying the presence or absence of cells and the kinds of cells by irradiating the micro flow path 250 with laser light from a light source 260 and detecting scattered light or fluorescence with a detector 255 using a condenser lens 265. In a case where this method is used, it is possible to estimate the number of cells that have landed in the predetermined well from the number of cells that have passed through the micro flow path 250.

Further, as the ejection head 10′ shown in FIG. 19, a single cell printer manufactured by Cytena Gmbh can be used. In FIG. 19, it is possible to estimate the number of cells that landed in the predetermined well by estimating that the cells 350″ in the vicinity of the nozzle part have been ejected based on the result obtained by analyzing an image of the vicinity of the nozzle part, where the image is acquired by an image acquisition part 255′ through a lens 265′ before ejection, or by estimating the number of cells that are considered to have been ejected based on the difference in cell number between images before and after the ejection. In the method for counting cells that have passed through the micro flow path, which is shown in FIG. 18, liquid droplets are continuously generated, whereas, in FIG. 19, liquid droplets can be formed on demand, which is more preferable.

<<Treatment for Counting Cells after Landing>>

As the treatment for counting cells after landing, it is possible to adopt a method for detecting fluorescently stained cells by observing the well in the plate or the insoluble carrier with a fluorescence microscope or the like. Such a method is described, for example, in Moon S., et al., Drop-on-demand single cell isolation and total RNA analysis, PLoS One, 6, (3), e17455, 2011.

The method for observing cells before the ejection of the liquid droplet and after the landing of the liquid droplet has the following problems, and thus it is most preferable to observe cells that are being ejected in the liquid droplet, depending on the kind of plate to be generated.

In the method for observing cells before ejection, the number of cells that are considered to have landed is measured based on the number of cells that have passed through the flow path and the observation of the images before and after ejection, and thus it is not checked whether the cells have been actually ejected, whereby an unexpected error may occur. For example, in a case where the nozzle unit is dirty, the liquid droplet is not ejected correctly and is attached to the nozzle plate, and thus the cells in the liquid droplet do not land. In addition, problems may occur such as cells remaining in a narrow region of the nozzle unit and cells moving more distantly than expected due to the ejection operation and going out of the observation range.

There is also a problem in the method for detecting cells on the plate after landing. First, it is necessary to prepare a plate with which microscopic observation can be performed. As the plate with which observation can be performed, a plate having a transparent and flat bottom surface, particularly a plate having a glass bottom surface is generally used. However, since such a plate is a special plate, there is a problem in that a general well cannot be used. In addition, in a case where the number of cells is as large as several tens thereof, there is a problem in that accurate counting cannot be performed because cells overlap.

Therefore, after ejecting the liquid droplet and before landing in the well or the insoluble carrier of the liquid droplet, it is preferable to perform a treatment for observing cells before ejection and a treatment for counting the cells after landing, in addition to measuring the number of cells contained in the liquid droplet by a sensor and a cell number-measuring means.

As the light-receiving element, a light-receiving element having one or a small number of light-receiving parts, for example, a photodiode, an avalanche photodiode, a photomultiplier tube can be used. In addition, it is also possible to use a sensor such as a charge-coupled device (CCD), a complementary metal-oxide-semiconductor (CMOS), or a gate CCD, in which light-receiving elements are arranged in two-dimensional array.

In a case where a light-receiving element having one or a small number of light-receiving parts is used, it is conceivable to determine how many cells are contained based on the fluorescence intensity by using a calibration curve prepared in advance. However, binary detection of the presence or absence of cells in the flying liquid droplet is mainly carried out. In a case where the ejection is performed in a state where the cell concentration of the cell suspension is sufficiently low and only 1 or 0 cells are contained in the liquid droplet, it is possible to perform counting with sufficient accuracy by binary detection.

In a case of assuming that cells are randomly arranged in the cell suspension, the number of cells in the flying liquid droplet is considered to follow the Poisson distribution, and the probability P (>2) that two or more cells will be contained in the liquid droplet is represented by Expression 4 below.

P(>2)=1−(1+λ)×e^{-λ} Expression 4

FIG. 20 is a graph showing the relationship between the probability P (>2) and the average cell number. Here, λ is the average cell number in the liquid droplet, which is obtained by multiplying the cell concentration in the cell suspension by the volume of the ejected liquid droplet.

In a case of measuring the number of cells by binary detection, it is preferable that the probability P (>2) be a sufficiently small value in order to ensure accuracy, and λ<0.15 is preferable, where the probability P (>2) is 1% or less. The light source is not particularly limited as long as it can induce the excitation of the fluorescence of cells, and can be appropriately selected depending on the intended purpose. A light source in which a general lamp such as a mercury lamp or halogen lamp is equipped with a filter for emitting a specific wavelength, a light-emitting diode (LED), a laser, or the like can be used. However, it is preferable to use a laser since it is necessary to irradiate a narrow region with light having high intensity, particularly in a case of forming a fine liquid droplet of 1 nL or less. As the laser light source, various generally known lasers such as a solid-state laser, a gas laser, and a semiconductor laser can be used. Further, the excitation light source may be one that continuously emits light to the region through which the liquid droplet passes or may be one that emits light in a pulsed manner at a timing at which a predetermined time delay is applied to the liquid droplet ejection operation in synchronization with the liquid droplet ejection.

(Step of Calculating Certainty of Number of Nucleic Acids Estimated in Cell Suspension Generation Step, Liquid Droplet-Landing Step, and Cell Number-Measuring Step)

This step is a step of calculating the certainty in each of the cell suspension generation step, the liquid droplet-landing step, and the cell number-measuring step. The calculation of the certainty of the estimated number of nucleic acids can be calculated in the same manner as the certainty in the cell suspension generation step.

Regarding the timing of the certainty calculation, the certainty may be collectively calculated in the next step of the cell number-measuring step or may be calculated by calculating uncertainty at the end of each of the cell suspension generation step, the liquid droplet-landing step, and the cell number-measuring step and by synthesizing each uncertainty in the next step of the cell number-measuring step. In other words, the certainty in each of the above steps may be appropriately calculated before the synthetic calculation.

(Output Step)

The output step is a step of outputting the value measured by the cell number-measuring means based on the detection result measured by the sensor as the number of cells contained in the cell suspension landed in a well or an insoluble carrier. The measured value means the number of cells contained in the well or the insoluble carrier, which is measured by the cell number-measuring means from the detection result measured by the sensor.

Output means that a device such as a motor, a communication device, or a computing machine receives an input and transmits the measured value as electronic information to an external server as a counting result storage means, or prints the measured value as a printed matter.

In the output step, at the time of generating a plate, the number of cells or the number of target nucleic acids in each well or the insoluble carrier in the plate is observed or estimated, and the observed value or the estimated value is output to an external memory unit. The output may be performed at the same time as the cell number-measuring step is performed or may be performed after the cell number-measuring step.

(Recording Step)

The recording step is a step of recording the observed output value or estimated value in the output step. The recording step can be preferably carried out in the recording unit. The recording may be performed at the same time as the output step is performed or after the output step. The recording includes not only adding information to a recording medium but also storing information in a recording unit.

(Nucleic Acid Extraction Step)

The nucleic acid extraction step is a step of extracting nucleic acid with an enzyme from cells in the well or the insoluble carrier. Extraction means destroying a cell membrane, a cell wall, or the like, and extracting a nucleic acid. The enzyme is not particularly limited as long as the nucleic acid can be extracted from cells; however, for example, in a case where the cell is yeast, Zymolyase or the like is preferable.

In a case of cells that have a cell wall, DNA may not be sufficiently extracted with the enzyme alone. In such a case, for example, an osmotic shock method, a freeze-thaw method, the use of a DNA extraction kit, a sonication method, a French press method, and a method of using a homogenizer may be used in combination.

(Enzyme Deactivation Step)

The enzyme deactivation step is a step of deactivating the enzyme used for extracting nucleic acid in the nucleic acid extraction step. In the method for manufacturing a device of the present embodiment, the deactivation of the enzyme is performed by drying at 5° C. to 45° C., preferably 10° C. to 45° C., more preferably 20° C. to 45° C., still more preferably 10° C. to 40° C., and particularly preferably at 20° C. to 40° C. The drying deactivation method is not particularly limited as long as it can deactivate the enzyme in the above temperature range, and the drying may be drying under atmospheric pressure (1 atm) or may be drying under reduced pressure, however, drying under reduced pressure is preferable. In a case where the enzyme is dried and deactivated in the above range, the nucleic acid can be reliably immobilized in the reaction field of the well, and the accuracy of the device can be secured even in a case where the nucleic acid immobilized in the reaction field is stored at room temperature. Regarding the storage, it is preferable to store the immobilized nucleic acid in a vacuum together with a desiccant such as silica gel.

(Other Steps)

Other steps are not particularly limited, and can be appropriately selected depending on the intended purpose. Examples thereof include a step of adding additional reagents.

Examples of the additional reagent include an intercalator, a primer, and an amplification reagent. The amplification reagent is the same as that described above.

[Device]

In one embodiment, the present invention provides a device that has at least one well, in which a specific number of copies of a nucleic acid in the at least one well are immobilized in a reaction field and the nucleic acid is extracted with an enzyme and immobilized in the reaction field by drying the enzyme at 5° C. to 45° C.

The device of the present embodiment is particularly suitable for application to the performance evaluation for a real-time PCR apparatus. Specifically, for example, the apparatus of the present embodiment can be applied to a quantitative PCR apparatus as a genetic examination apparatus.

The device of the present embodiment has at least one well, in which a specific number of copies of a nucleic acid in the at least one well are immobilized in a reaction field and the nucleic acid is extracted with an enzyme and immobilized in the reaction field by drying the enzyme at 5° C. to 45° C. In addition to a specified number of copies of a nucleic acid, the device of the present embodiment may contain additional reagents that allow the amplification reaction.

Examples of the additional reagents include a primer and amplification reagent. The primer is a synthetic oligonucleotide having a base sequence of 18 to 30 bases complementary and specific to the template DNA in the polymerase chain reaction (PCR), and a pair of a forward primer (a sense primer) and a reverse primer (an antisense primer) are set at two places so that a target region to be amplified is sandwiched.

Examples of the amplification reagent in the polymerase chain reaction (PCR) include a DNA polymerase as an enzyme, four kinds of bases (dGTP, dCTP, dATP, and dTTP) as substrates, Mg²⁺ (magnesium chloride at a final concentration of about 2 mM), and a buffer for maintaining the optimum pH (pH 7.5 to 9.5).

The nucleic acid in the well is extracted with an enzyme and immobilized in the reaction field by drying the enzyme at 5° C. to 45° C. In a case where the nucleic acid in the well is extracted with an enzyme, and then the enzyme is dried at 5° C. to 45° C., the nucleic acid can be reliably immobilized in the reaction field, and the accuracy of the device can be secured even in a case where the nucleic acid is stored at room temperature. In addition, in a case where a reagent (hereinafter, also referred to as an “amplification reagent”) that enables the amplification reaction is added, the nucleic acid immobilized in the reaction field elutes from the reaction field, and it is possible to more accurately evaluate a PCR reaction from the specific number of nucleic acids.

It is preferable that the well contain an appropriate amount of the reagent so that the reagent can be instantly used as a reaction solution by dissolving the reagent in the solid dry state in a buffer or water immediately before using the device.

In the device of the present embodiment, a specified number of copies of a nucleic acid may be immobilized in the reaction field in all of the plurality of wells, or the specified number of copies of a nucleic acid may be immobilized in the reaction field in some of the plurality of wells. In the latter case, the remaining wells, for example, may be empty or may contain a reagent having different compositions. The specified copy number is as described above.

In the device of the present embodiment, the form of the well is not particularly limited, and for example, the device of the present embodiment may have a form of a well plate.

In the device of the present embodiment, the immobilization of the nucleic acid to the reaction field may be the direct immobilization to the reaction field or may be the addition of an insoluble carrier, on which a specified number of copies of a nucleic acid are immobilized, to the reaction field.

In a case where an insoluble carrier, on which a specified number of copies of a nucleic acid are immobilized, is added to the reaction field, the insoluble carrier, on which a specified number of copies of a nucleic acid is immobilized, can be added in the reaction field of the well as the whole insoluble carrier, which makes it possible to select a reaction field in which a specified number of copies of a nucleic acid are reacted.

The material of the insoluble carrier is not particularly limited as long as it is insoluble in the reaction solution, and can be appropriately selected depending on the intended purpose. Examples thereof include polystyrene, polypropylene, polyethylene, fluororesin, an acrylic resin, polycarbonate, polyurethane, polyvinyl chloride, polyethylene terephthalate, and a cyclic olefin copolymer (COC).

(Well)

The shape, number, volume, material, color, and the like of the well are not particularly limited and can be appropriately selected according to the intended purpose. The shape of the well is not particularly limited as long as it is possible to contain a specified number of copies of a nucleic acid and an intercalator, and in a case of being present, additional reagents, and can be appropriately selected depending on the intended purpose. Examples thereof include a flat bottom and a recessed part such as a round bottom, a U-shaped bottom, or a V-shaped bottom.

The number of wells is plural, preferably 5 or more, and more preferably 50 or more. A multi-well plate having two or more wells is preferably used. Examples of the multi-well plate include a well plate having 24, 48, 96, 384, or 1,536 wells.

The volume of the well is not particularly limited and can be appropriately selected depending on the intended purpose; however, considering the amount of sample that is used in the general real-time PCR apparatus, 10 μL or more and 1,000 μL or less is preferable.

The material of the well is not particularly limited and can be appropriately selected depending on the intended purpose. Examples thereof include polystyrene, polypropylene, polyethylene, fluororesin, an acrylic resin, polycarbonate, polyurethane, polyvinyl chloride, and polyethylene terephthalate.

Regarding the color, the well may be, for example, transparent, translucent, colored, or completely light-shielded. The wettability of the well is not particularly limited and may be appropriately selected depending on the intended purpose and, for example, may be a water repellency. In a case where the wettability of the well is a water repellency, the adsorption of the reagent to the inner wall of the well can be reduced. Further, in a case where the wettability of the well is a water repellency, it is easy to move the reagent in the well in a solution state.

The method of making the inner wall of the well water repellent is not particularly limited and may be appropriately selected depending on the intended purpose. Examples thereof include a method of forming a fluorine-based resin film, a fluorine plasma treatment, and embossing processing. In particular, in a case of a water repellency treatment providing a contact angle of 100° or more, it is possible to suppress a decrease in reagent amount, uncertainty, and an increase in the coefficient of variation due to liquid spillage.

(Base Material)

The device of the present embodiment is preferably a plate-shaped device having wells provided on a base material; however, it may be a connection type well tube such as an 8-well strip tube. The base material is not particularly limited in terms of material, shape, size, structure, and the like, and can be appropriately selected depending on the intended purpose.

The material of the base material is not particularly limited and may be appropriately selected depending on the intended purpose. Examples thereof include semiconductors, ceramics, metals, glass, quartz glass, and plastics. Among these, plastics are preferable.

Examples of the plastics include polystyrene, polypropylene, polyethylene, fluororesin, an acrylic resin, polycarbonate, polyurethane, polyvinyl chloride, and polyethylene terephthalate.

The shape of the base material is not particularly limited, and can be appropriately selected depending on the intended purpose. Examples thereof include a sheet shape and a plate shape. The structure of the base material is not particularly limited and may be appropriately selected depending on the intended purpose. For example, it may be a single-layer structure or may be a multi-layer structure.

(Identification Means)

The device of the present embodiment may have an identification means capable of identifying information on the specific copy number of the nucleic acid. The identification means is not particularly limited and may be appropriately selected depending on the intended purpose. Examples thereof include a memory, an IC chip, a barcode, a QR code (registered trade mark), a radio-frequency identifier (hereinafter, also may be referred to as an “RFID”), color coding, and printing.

The position where the identification means is provided and the number of identification means are not particularly limited and can be appropriately selected depending on the intended purpose.

In addition to the information on the specific copy number of the nucleic acid, examples of the information stored in the identification means include analysis results (for example, the Cq value and the scattering of Cq values), the number (for example, the number of cells) of nucleic acids arranged in the well, cell survival and cell death, the information on the well that is filled with nucleic acid among the plurality of wells, the kind of nucleic acid, the date and time of measurement, and the name of a measurer.

The information stored in the identification means can be read by using various reading means. For example, in a case where the identification means is a barcode, a barcode reader is used as the reading means.

The method of writing information in the identification means is not particularly limited and may be appropriately selected depending on the intended purpose. Examples thereof include manual inputting, a method of directly writing data from a liquid droplet-forming device, where the number of nucleic acids is counted when dispensing the nucleic acid into a well, the transferring of data stored in the server, and the transferring of data stored in the cloud.

(Sealing Member)

Other members are not particularly limited, and can be appropriately selected depending on the intended purpose. Examples thereof include a sealing member.

The device of the present embodiment preferably has a sealing member in order to prevent foreign matter from entering the well or a filling material from flowing out of the well. The sealing member is configured to seal at least one well and may be configured to be capable of being cut and detached along a cutting line so that the sealing member can be individually sealed or opened.

Examples of the shape of the sealing member include a cap shape that matches the diameter of the inner wall of the well and a film shape that covers the well opening portion.

In a case where the device of the present embodiment has a sealing member, the reaction field in which a nucleic acid is immobilized is not particularly limited as long as it is a specific reaction space in the well, and the reaction field may be the bottom surface of the well or may be a surface of an insoluble carrier serving as the sealing member, where the surface comes into contact with the reaction field. For example, it may be a surface (a back surface) of a cap that comes into contact with the inside of the well and matches the diameter of the inner wall of the well. In a case where the nucleic acid is immobilized on a surface of an insoluble carrier serving as the sealing member, where the surface comes into contact with the reaction field, it is necessary to release the nucleic acid immobilized on the surface of the insoluble carrier, where the surface comes into contact with the reaction field, from the insoluble carrier when the nucleic acid reacts. Therefore, in a case where the nucleic acid is immobilized to an insoluble carrier serving as the sealing member, the nucleic acid immobilized to the insoluble carrier is released, for example, by the inversion mixing of the well sealed by the sealing member.

In a case where the nucleic acid is immobilized on a surface of the insoluble carrier serving as the sealing member, where the surface comes into contact with the reaction field, any well can be sealed with a sealing member on which the nucleic acid is immobilized, and thus it is possible to carry the nucleic acid and select a reaction field in which the nucleic acid is reacted.

Examples of the material of the insoluble carrier serving as the sealing member include a polyolefin resin, a polyester resin, a polystyrene resin, and a polyamide resin. The sealing member preferably has a film shape with which all wells can be sealed at one time. Further, the sealing member may be configured so that the adhesive strength to a well that needs to be resealed is different from that to a well that does not need to be resealed, thereby reducing misuse by a user.

In the device of the present embodiment, the specific copy number of the nucleic acid immobilized in a reaction field of one well and the specific copy number of the nucleic acid immobilized in reaction fields of other wells may be the same in all the wells or may be two or more copy numbers different from each other. In the former case, examples of the case of the specific copy number include, in terms of the copy number of all wells, a case of 1 copy, a case of 5 copies, a case of 10 copies, a case of 20 copies, a case of 40 copies, a case of 80 copies, a case of 160 copies, and a case of 200 copies. In the latter case, examples of the specific copy number include a case of 1, 5, 20, 40, 80, 160, and 200, a case of 1, 2, 3, 4, 5, 6, 7, 8, 9, 10, a case of 1, 3, 5, 7, and 9, and a case of 2, 4, 6, 8, and 10. Further, some of the wells may not contain nucleic acid and may be used as the negative control.

With the device in which the specific copy number of the nucleic acid immobilized in a reaction field of one well and the specific copy number of the nucleic acid immobilized in a reaction field of other wells are the same in all the wells, it is easy to compare the evaluation results of the amplification reaction between wells. As a result, it can be suitably used in the performance evaluation method for the real-time PCR apparatus described above.

Further, in the device of the present embodiment, the specific copy number of the nucleic acid immobilized in the reaction field of one well may be 10¹¹ and the specific copy number of the nucleic acid immobilized in the reaction field of other wells may be 10^{N2} (here, N1 and N2 are consecutive integers). Specific examples of the case of the copy number include cases of 1, 10, 100, 1,000, 100, 1,000, 10,000, 100,000, and 1,000,000.

The device of the present embodiment may have groups of two or more wells having a different specific copy number of the nucleic acid immobilized in the reaction field. For example, in a case where the base material of the device is a plate having a plurality of wells, each group forms each group “region” on the plate. In the “regions” formed by two or more groups having different specific copy numbers of the nucleic acid, the wells may be adjacent to each other or separated from each other.

As a result, based on the results obtained by performing real-time PCR using the device of the present embodiment, for example, in a case where wells at different positions, having the same specific copy number, are compared and there is a well (an inadequate well) that is not suitable for use, it is possible to determine whether to calibrate the real-time PCR apparatus again or to exclude a sample in the non-inadequate well in the actual sample measurement.

(Nucleic Acid)

The nucleic acid means a high-molecular-weight organic compound in which nitrogen-containing bases derived from purine or pyrimidine, sugar, and phosphoric acid are regularly bonded, and includes a nucleic acid analog and the like. The nucleic acid is not particularly limited, and can be appropriately selected depending on the intended purpose. Examples thereof include DNA, RNA, and cDNA. The nucleic acid may be a fragment of a nucleic acid or may be incorporated into the nucleus of the cell; however, it is preferably incorporated into the nucleus of the cell.

The nucleic acid may be a natural product obtained from an organism, a processed product thereof, a nucleic acid produced using genetic recombination technology, or an artificially synthesized nucleic acid that is chemically synthesized. One kind of these may be used alone, or two or more kinds thereof may be used in combination. In a case where an artificially synthesized nucleic acid is used, impurities can be reduced and the molecular weight can be reduced, and thus the initial reaction efficiency can be improved.

The artificially synthesized nucleic acid means a nucleic acid obtained by an artificial synthesis, which is composed of the same constitutional components (base, deoxyribose, phosphoric acid) as those of the naturally occurring DNA or RNA. The artificially synthesized nucleic acid may be, for example, a nucleic acid having a base sequence encoding a protein or may be a nucleic acid having any base sequence.

Examples of the nucleic acid analog or nucleic acid fragment analog include a nucleic acid or nucleic acid fragment to which a non-nucleic acid component is bound, or a nucleic acid or nucleic acid fragment (for example, a primer or a probe labeled with a fluorescent dye or a radioisotope) which is labeled with a labeling agent such as a fluorescent dye or an isotope, and an artificial nucleic acid (for example, PNA, BNA, or LNA) in which the chemical structure of some of the nucleotides constituting the nucleic acid or nucleic acid fragment is changed.

The form of the nucleic acid is not particularly limited, and can be appropriately selected depending on the intended purpose. Examples thereof include a double-stranded nucleic acid, a single-stranded nucleic acid, and a partially double-stranded or single-stranded nucleic acid, and may be a circular or linear plasmid. In addition, the nucleic acid may have a modification or a mutation.

The nucleic acid preferably has a specific base sequence which is clearly revealed. The specific base sequence is not particularly limited, and can be appropriately selected depending on the intended purpose. Examples thereof include a base sequence that is used for an infectious disease examination, a non-natural base sequence that does not exist in nature, a base sequence derived from an animal cell, a base sequence derived from a plant cell, a base sequence derived from a fungal cell, a base sequence derived from a bacterium, and a base sequence derived from a virus. One kind of these may be used alone, or two or more kinds thereof may be used in combination.

In a case where an unnatural base sequence is used, the GC content of the base sequence is preferably 30% or more and 70% or less, and the GC content is preferably fixed. The base length of the nucleic acid is not particularly limited and may be appropriately selected depending on the intended purpose. Examples thereof include a base length of 20 base pairs (or mer) or more and 10,000 base pairs (or mer) or less.

In a case where a base sequence that is used for an infectious disease examination is used as the nucleic acid, the base sequence is not particularly limited as long as it contains a base sequence unique to the infectious disease, and can be appropriately selected depending on the intended purpose; however, it preferably contains the base sequence specified by the official method or the notified method.

The nucleic acid may be a nucleic acid derived from the cell to be used or may be a nucleic acid introduced by gene transfer. The kind of nucleic acid may be one or more kinds. In a case where a nucleic acid incorporated into the nucleus of a cell by gene transfer is used as the nucleic acid, it is preferable to confirm that a specific number of copies (for example, 1 copy) of a nucleic acid are introduced into one cell. The method for confirming that a specific number of copies of a nucleic acid have been introduced is not particularly limited, and can be appropriately selected depending on the intended purpose. Examples thereof include sequencing, a PCR method, and Southern blotting.

In a case of introducing a nucleic acid into the nucleus of a cell, the method for gene transfer is not particularly limited as long as the desired number of copies of a specific nucleic acid sequence can be introduced into the target location. Examples thereof include homologous recombination, CRISPR/Cas9, CRISPR/Cpf1, TALEN, Zinc finger nuclease, Flip-in, and Jump-in. Alternatively, the nucleic acid may be introduced into the nucleus of the cell in the form of a plasmid, artificial chromosome, or the like.

For example, in a case where yeast (a yeast cell) is used as the cell, homologous recombination is preferable among them from the viewpoint of high efficiency and ease of control.

(Carrier)

The nucleic acid is preferably handled in a state of being supported on a carrier. For example, an aspect in which the nucleic acid is supported on (more preferably encompassed in) a particle-shaped carrier (a carrier particle) can be mentioned. The carrier is not particularly limited and may be appropriately selected depending on the intended purpose, and examples thereof include a cell, a resin, a liposome, and a microcapsule.

<<Cell>>

The cell is a structural and functional unit that forms an organism, and a specific sequence in the nucleus can be used as the nucleic acid. The nucleic acid may be a base sequence that originally exists in the nucleus or may be introduced from the outside.

The cell is not particularly limited, and can be appropriately selected depending on the intended purpose. Examples thereof include a eukaryotic cell, a prokaryotic cell, a cell of a multicellular organism, and a cell of a unicellular organism. One kind of cell may be used alone, or two or more thereof may be used in combination.

The eukaryotic cell is not particularly limited, and can be appropriately selected depending on the intended purpose. Examples thereof include an animal cell, an insect cell, a plant cell, a fungal cell, algae, and protozoa. One kind of these may be used alone, or two or more kinds thereof may be used in combination. Among these, an animal cell or a fungal cell is preferable.

The animal cell may be an adhesive cell or a floating cell. The adhesive cell may be a primary cell collected directly from tissues or organs, or may be a passaged cell of the primary cell collected directly from tissues or organs for several generations, may be a differentiated cell, or may be an undifferentiated cell.

The differentiated cell is not particularly limited, and can be appropriately selected depending on the intended purpose. Examples thereof include an endothelial cell such as a hepatocyte, which is a parenchymal cell of the liver, a stellate cell, a Kupffer cell, a vascular endothelial cell, a sinusoidal endothelial cell, or a corneal endothelial cell; an epidermal cell such as a fibroblast, an osteoblast, an osteoclast, a periodontal ligament-derived cell, or an epidermal keratinocyte; an epithelial cell such as a tracheal epithelial cell, a gastrointestinal epithelial cell, a cervical epithelial cell, or a corneal epithelial cell; a mammary gland cell, a pericyte; a muscle cell such as a smooth muscle cell or a myocardial cell, a renal cell, a pancreatic islet of Langerhans cell; a nerve cell such as a peripheral nerve cell or an optic nerve cell, and a cartilage cell and a bone cell.

The undifferentiated cell is not particularly limited, and can be appropriately selected depending on the intended purpose. Examples thereof include a totipotent stem cell such as an embryonic stem cell (an ES cell) or an induced pluripotent stem cell (an iPS cell); a pluripotent stem cell such as a mesenchymal stem cell; and a unipotent stem cell such as a vascular endothelial precursor cell.

The fungal cell is not particularly limited, and can be appropriately selected depending on the intended purpose. Examples thereof include molds and yeasts. One kind of these may be used alone, or two or more kinds thereof may be used in combination. Among them, yeast is preferable since the cell cycle can be regulated and a haploid can be used. The cell cycle means the period in which cell division occurs when cells proliferate, and cells (daughter cells) generated by cell division become cells (mother cells) that undergo cell division again to produce new daughter cells.

The yeast is not particularly limited, and can be appropriately selected depending on the intended purpose. For example, the yeast is preferably one that has been synchronously cultured in the G0/G1 phase and arrested in the G1 phase. Further, the yeast is, for example, preferably a Bar-1 gene-deficient yeast having increased sensitivity to a pheromone (a sex hormone) that controls the cell cycle in the G1 phase. In a case where the yeast is a Bar-1 gene-deficient yeast, the abundance ratio of the yeast whose cell cycle cannot be controlled can be reduced, and thus an increase in the number of nucleic acids in the cells accommodated in the well or the like can be prevented.

The prokaryotic cell is not particularly limited, and can be appropriately selected depending on the intended purpose. Examples thereof include eubacteria such as Escherichia coli and archaea. One kind of these may be used alone, or two or more kinds thereof may be used in combination.

The cell is preferably a dead cell. In a case of the dead cell, cell division can be prevented from occurring after isolation. It is preferable that the cell can emit light in a case where receiving light. In a case where the cells are capable of emitting light in when receiving light, it is possible to cause the cells to land in the well while the number of cells is controlled with high accuracy.

It is preferable that the cell can emit light when receiving light. Light receiving means receiving light. Light emission by a cell is detected by an optical sensor. The optical sensor means a passive type sensor that collects, with a lens, any one of visible light that can be seen by the human eye, near infrared light having a longer wavelength than visible light, short wavelength infrared light, and light up to the thermal infrared light region, and then acquires the shape or the like of the cell of interest as image data.

The cell capable of emitting light when receiving light is not particularly limited, and can be appropriately selected depending on the intended purpose. Examples thereof include a cell stained with a fluorescent dye, a cell expressing a fluorescent protein, and a cell labeled with a fluorescently labeled antibody. The portion stained with the fluorescent dye, the portion expressing the fluorescent protein, and the portion labeled with the fluorescently labeled antibody in the cell is not particularly limited, and examples thereof include the whole cell, cell nucleus, and cell membrane.

Examples of the fluorescent dye include fluoresceins, azos, rhodamines, coumarins, pyrenes, and cyanines. One kind of these may be used alone, or two or more kinds thereof may be used in combination. Among them, fluoresceins, azos, rhodamines, or cyanines are preferable, and eosin. Evans blue, trypan blue, rhodamine 6G, rhodamine B, rhodamine 123, or Cy3 is more preferable.

As the fluorescent dye, a commercially available product can be used. Examples of the commercially available product include product name: Eosin Y (manufactured by FUJIFILM Wako Pure Chemical Corporation), product name: Evans Blue (manufactured by FUJIFILM Wako Pure Chemical Corporation), product Name: Trypan Blue (manufactured by FUJIFILM Wako Pure Chemical Corporation), Product name: Rhodamine 6G (manufactured by FUJIFILM Wako Pure Chemical Corporation), Product name: Rhodamine B (manufactured by FUJIFILM Wako Pure Chemical Corporation), and product name: Rhodamine 123 (manufactured by FUJIFILM Wako Pure Chemical Corporation).

Examples of the fluorescent protein include Sirius, EBFP, ECFP, mTurquoise, TagCFP, AmCyan, mTFPI, MidoriishiCyan, CFP, TurboGFP, AcGFP, TagGFP, Azami-Green, ZsGreen, EmGFP, EGFP, GFP2, HyPer, TagYFP, EYFP, Venus, YFP, PhiYFP, PhiYFP-m, TurboYFP, ZsYellow, mBanana, KusabiraOrange, mOrange, TurboRFP, DsRed-Express, DsRed2, TagRFP, DsRed-Monomer, AsRed2, mStrawberry, TurboFP602, mRFP1, JRed, KillerRed, mCherry, mPlum, PS-CFP, Dendra2, Kaede, EosFP, and KikumeGR. One kind of these may be used alone, or two or more kinds thereof may be used in combination.

The fluorescently labeled antibody is not particularly limited as long as it can bind to the target cell and is fluorescently labeled, and can be appropriately selected depending on the intended purpose. Examples thereof include a FITC-labeled anti-CD4 antibody and a PE-labeled anti-CD8 antibody. One kind of these may be used alone, or two or more kinds thereof may be used in combination.

The volume-average cell size of cells is preferably 30 μm or less, more preferably 10 μm or less, and particularly preferably 7 μm or less in the free state. In a case where the volume-average cell size is 30 μm or less, cells can be suitably used for a liquid droplet ejection means, such as an inkjet method or a cell sorter.

The volume-average cell size of cells can be measured by, for example, the following measuring method. In a case where yeast is used as cells, 10 μL of the prepared dispersion solution of the stained yeast is taken out, placed on a plastic slide made of PMMA, and the volume-average cell size can be measured by using an automatic cell counter (trade name: Countess Automated Cell Counter, manufactured by Invitrogen) or the like. The number of cells can also be determined by the same measuring method.

The density of cells in the cell suspension is not particularly limited, and can be appropriately selected depending on the intended purpose, but the density is preferably 5×10⁴ cells/mL or more and 5×10⁸ cells/mL or less and more preferably 5×10⁴ cells/mL or more and 5×10⁷ cells/mL or less. In a case where the cell density is in the above range, the ejected liquid droplets can reliably contain cells. The cell density can be measured using an automatic cell counter (trade name: Countess Automated Cell Counter, manufactured by Invitrogen) or the like in the same manner as the measuring method for the volume-average cell size.

<<Resin>>

The resin is not particularly limited in terms of material, shape, size, and structure, as long as a nucleic acid can be supported, and can be appropriately selected depending on the intended purpose.

<<Liposome>>

The liposome is a lipid vesicle formed from a lipid bilayer containing lipid molecules and specifically means a closed lipid-containing vesicle, which has a space separated from the outside by a lipid bilayer formed based on the polarities of the hydrophobic group and hydrophilic group of the lipid molecule.

The liposome is a closed vesicle formed by a lipid bilayer using lipids and has an aqueous phase (an inner aqueous phase) in the space of the closed vesicle. The inner aqueous phase contains water and the like. The liposomes may be a single lamella (a single-layer lamella, a uni-lamella, a single-layered bilayer membrane) or a multi-layered lamella (a multi-lamella, multiple bilayer membranes having an onion-like structure, where each layer is partitioned by a watery layer).

The liposome preferably encapsulate nucleic acid, and the form thereof is not particularly limited. “Encapsulation” means that the nucleic acid is contained in the inner aqueous phase or the membrane itself with respect to the liposome. Examples of the form thereof include a form in which a nucleic acid is encapsulated in a closed space formed by the membrane and a form in which a nucleic acid is encapsulated in the membrane itself, and a combination thereof may be used.

The size (the average particle size) of the liposome is not particularly limited as long as a nucleic acid can be encapsulated. In addition, the shape thereof is preferably a spherical shape or a shape similar thereto.

The component (the membrane component) constituting the lipid bilayer of the liposome is selected from lipids. As the lipid, any lipid can be used as long as it is soluble in a mixed solvent of a water-soluble organic solvent and an ester-based organic solvent. Specific examples of the lipid include a phospholipid, a lipid other than the phospholipid, cholesterols, and derivatives thereof. These components may be used alone or in a combination of two or more kinds thereof.

<<Microcapsule>>

The microcapsule means a fine particle having a wall material and a hollow structure and a nucleic acid can be encapsulated in the hollow structure. The microcapsule is not particularly limited, and the wall material, the size, and the like can be appropriately selected according to the intended purpose.

Examples of the wall material of the microcapsule include a polyurethane resin, polyurea, a poly urea-polyurethane resin, a urea-formaldehyde resin, a melamine-formaldehyde resin, polyamide, polyester, polysulfone amide, polycarbonate, polysulfinate, an epoxy, acrylic acid ester, methacrylic acid ester, vinyl acetate, and gelatin. One kind of these may be used alone, or two or more kinds thereof may be used in combination.

The size of the microcapsule is not particularly limited as long as a nucleic acid can be encapsulated, and can be appropriately selected depending on the intended purpose. The method for producing a microcapsule is not particularly limited, and can be appropriately selected depending on the intended purpose. Examples thereof include an in-situ method, an interfacial polymerization method, and a coacervation method.

The device of the present embodiment can be widely used in the biotechnology-associated industry, the life science industry, the medical industry, and the like, and can be suitably used for, for example, the performance evaluation and the quality control of a real-time PCR apparatus. In addition, it can be applied to methods specified by an official method, a notified method, and the like in infectious disease examination.

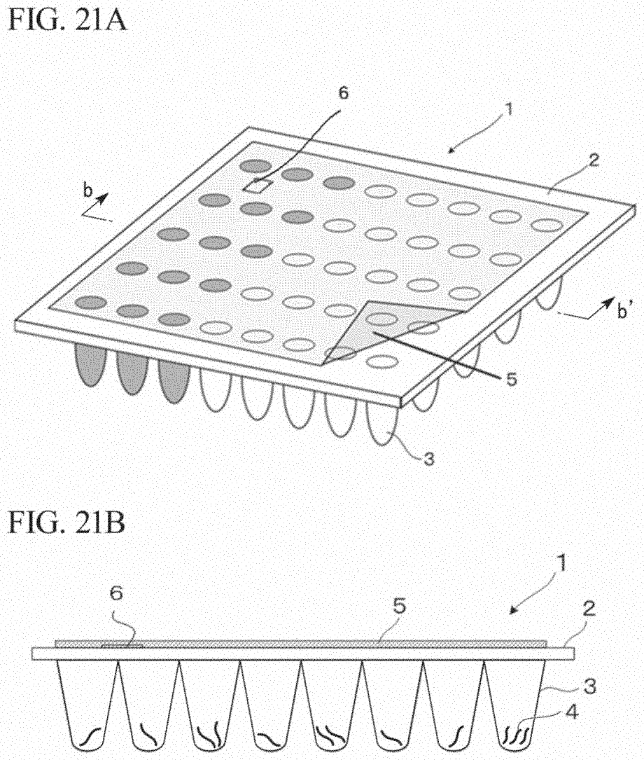

FIG. 21A is a perspective view showing an example of a device of the present embodiment. FIG. 21B is across-sectional view taken along the line b-b′ in the arrow direction in FIG. 21A.

A device 1 has a base material 2 and a plurality of wells 3 formed on the base material 2, and a specific number of copies of a nucleic acid 4 are immobilized on the reaction field (for example, the bottom surface) of the well 3. In Examples of FIG. 21A and FIG. 21B, the opening portion of the well is covered by a sealing member 5.

In addition, for example, an IC chip or a barcode (an identification means 6), which stores information on the specific copy number of the nucleic acid 4 immobilized on the reaction field of each well 3 and other information, is positioned at a position other than the opening portion of the well, between the sealing member 5 and the base material 2. Since the identification means 6 is positioned at this position, it is possible to prevent, for example, an unintended modification of the identification means 6. In addition, since the device 1 has the identification means 6, it can be distinguished from a general well plate which does not have the identification means 6. This makes it possible to prevent a device from being mistaken.

[Performance Evaluation Kit for Real-Time PCR Apparatus]

In one embodiment, the present invention provides a performance evaluation kit for a real-time PCR apparatus, including a device that has at least one well, in which a specific number of copies of a nucleic acid in the at least one well are immobilized in a reaction field and the nucleic acid is extracted with an enzyme and immobilized in the reaction field by drying the enzyme at 5° C. to 45° C.

The kit of the present embodiment is particularly suitable for application to the performance evaluation for the real-time PCR apparatus described above and can be suitably used for the performance evaluation method for the real-time PCR apparatus described above. In the kit of the present embodiment, the well, the specified copy number, the nucleic acid, the device, the reaction field, the enzyme, the drying, and the like are the same as those described above.

[Performance Evaluation Device and Performance Evaluation Program for Real-Time PCR Apparatus]

In one embodiment, the present invention provides a performance evaluation device for a real-time PCR apparatus, which has an information acquisition unit that acquires information on a nucleic acid amplification reaction by carrying out the amplification reaction using the device described above and an evaluation unit that evaluates the performance of a real-time PCR apparatus based on the information on the information acquisition unit, and, as necessary, further having another unit.

In one embodiment, the present invention provides a performance evaluation program for a real-time PCR apparatus, which causes a computer to carry out the processing of evaluating the performance of a real-time PCR apparatus based on the information on the amplification reaction, obtained by carrying out the amplification reaction using the device described above.

Since the controlling carried out by the control unit or the like in the performance evaluation device of the present embodiment is synonymous with carrying out the performance evaluation method for the real-time PCR apparatus described above, the details of the performance evaluation method for a real-time PCR apparatus will also be clarified through the explanation of the performance evaluation device of the present embodiment. In addition, since the performance evaluation program of the present embodiment realizes the performance evaluation device for a real-time PCR apparatus by using a computer or the like as the hardware resource, the details of the performance evaluation program for a real-time PCR apparatus of the present embodiment will also be clarified through the explanation of the performance evaluation device of the present embodiment.

(Amplification Reaction Information Acquisition Step and Information Acquisition Unit)

The step of evaluating the amplification reaction is a step of acquiring information on the amplification reaction using the above-described device and is carried out by the information acquisition unit. The information on the amplification reaction can be obtained by carrying out real-time PCR using the above-described device.

Examples of the amplification reaction information include Cq values and scattering of Cq values. One of these pieces of information may be used alone for evaluation, or two or more thereof may be used in combination for evaluation. The scattering of the Cq values is the same as that described above. Examples of the scattering of the Cq values include a standard deviation and a CV value.

(Evaluation Step and Evaluation Unit)

The evaluation step is a step of evaluating the performance of a real-time PCR apparatus based on the information on the amplification reaction and is carried out by the evaluation unit.

For example, in the qualitative evaluation, the Cq value may be measured by carrying out real-time PCR using the above-described device to calculate the average Cq value. The in-plane characteristics can be evaluated as “∘” in a case where the Cq value of each well is within 10% of the average Cq value and “x” in a case where the Cq value of each well is more than 10% of the average Cq value.

In addition, it is possible to obtain a chronological change in the information on the amplification reaction by using the device of the present embodiment and carrying out measurement for a predetermined period of time. As a result, similar to the in-plane characteristics, for example, in a case where the Cq value of each well is more than 10% of the average Cq value, it is possible to calibrate the examination apparatus or take measures not to use the measured place of the well. In addition, since the arranged specific copy number is an absolute value, it is possible to compare the performances between examination apparatuses in a case where devices in which an identical specific number of copies are arranged are used.

In the quantitative evaluation, it is possible to obtain a chronological change in the information on the amplification reaction by using the device of the present embodiment and carrying out measurement for a predetermined period of time. As a result, similar to the in-plane characteristics, for example, in a case where a value that deviates from the quality control value is obtained, it is possible to calibrate the examination apparatus or take measures not to use the measured place of the well. In addition, since the arranged copy number is an absolute value, it is possible to compare the performances between examination apparatuses in a case where devices in which an identical number of copies are arranged are used.

Further, in the case of quantitative evaluation, since it is possible to determine, for example, a copy number (a copy number or concentration) corresponding to the Cq value from the calibration curve and the PCR efficiency, instead of the Cq value itself, the performances between examination apparatuses may be evaluated using the copy number (the copy number or concentration), a CV value converted to copy number (a copy number or concentration),(Max−Min)/(2×average value)×100 of the copy number (the copy number or concentration converted), or the like.

(Other Steps and Other Units)

Other steps and other units are not particularly limited, and can be appropriately selected depending on the intended purpose. Examples thereof include a displaying step and a display unit.

The processing by the performance evaluation program of the present embodiment can be executed by using a computer having a control unit constituting the performance evaluation device. The hardware configuration and the functional configuration of the performance evaluation device will be described below.

(Hardware Configuration of Performance Evaluation Device)

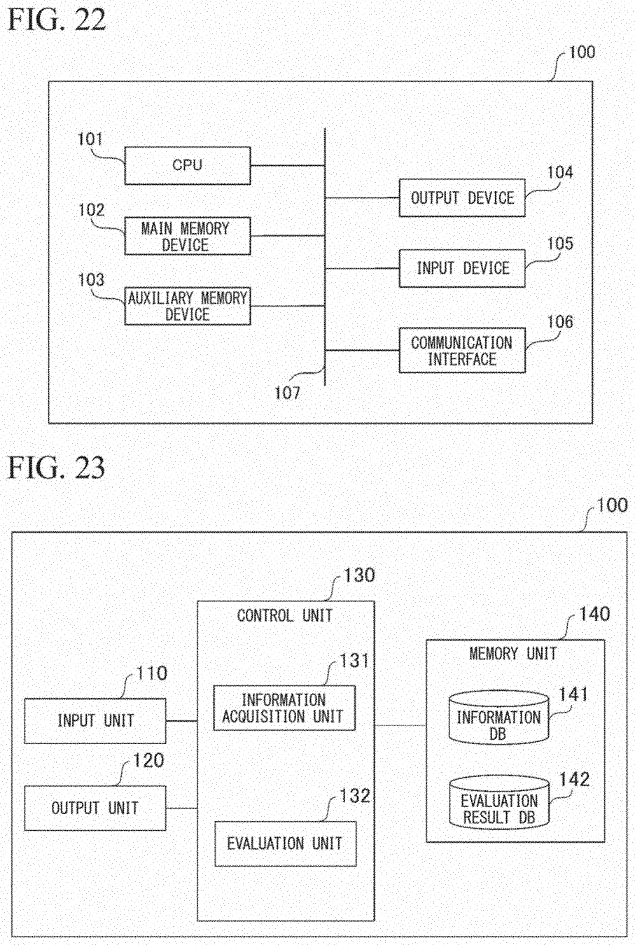

FIG. 22 is a block diagram showing an example of a hardware configuration of a performance evaluation device 100 of a real-time PCR apparatus. As shown in FIG. 22, the performance evaluation device 100 includes a central processing unit (CPU) 101, a main memory device 102, an auxiliary memory device 103, an output device 104, an input device 105, and a communication interface (a communication I/F) 106. Each of these is connected through a bus 107.

The CPU 101 is a processing device that performs various controls and calculations. The CPU 101 executes an operating system (OS) or a program, which is stored in the main memory device 102 or the like, thereby realizing various functions. That is, the CPU 101 executes the performance evaluation program for a real-time PCR apparatus, thereby functioning as a control unit 130 of the performance evaluation device 100 of the real-time PCR apparatus.

Further, the CPU 101 controls the operation of the performance evaluation device 100 of the examination apparatus on the whole. Here, the device that controls the operation of the performance evaluation device 100 on the whole is the CPU 101; however, the device is not limited to this and may be, for example, a field-programmable gate array (FPGA) or the like.

The performance evaluation program of the examination apparatus and various databases are not necessarily stored in the main memory device 102, the auxiliary memory device 103, or the like. The performance evaluation program of the examination apparatus and various databases may be stored in other information-processing apparatuses that are connected to the performance evaluation device 100 of the examination apparatus through the Internet, the local area network (LAN), the wide area network (WAN), or the like. The performance evaluation device 100 of the examination apparatus may be configured to acquire and execute the performance evaluation program of the examination apparatus and various databases from these other information-processing apparatuses.

The main memory device 102 stores various programs and stores data and the like necessary for executing various programs. The main memory device 102 has a read-only memory (ROM) and a random-access memory (a RAM), which are not shown in the drawing.

The ROM stores various programs such as the basic input/output system (BIOS). The RAM functions as a work area to be developed when various programs stored in the ROM are executed by the CPU 101. The RAM is not particularly limited and may be appropriately selected depending on the intended purpose. Examples of the RAM include a dynamic random-access memory (DRAM) and a static random-access memory (SRAM).

The auxiliary memory device 103 is not particularly limited as long as it can store various types of information, and can be appropriately selected depending on the intended purpose. Examples thereof include a solid-state drive and a hard disk drive.

Further, the auxiliary memory device 103 may be a portable memory device such as a compact disc (CD) drive, a digital versatile disc (DVD) drive, or a Blu-ray disc (BD) (registered trademark) drive.

As the output device 104, a display, a speaker, or the like can be used. The display is not particularly limited, and a known display can be appropriately used. Examples thereof include a liquid crystal display and an organic EL display.

The input device 105 is not particularly limited as long as it can accept various requests for the performance evaluation device 100 of the examination apparatus, and a known input device can be appropriately used. Examples thereof include a keyboard, a mouse, and a touch panel.

The communication interface (the communication I/F) 106 is not particularly limited, and a known communication interface can be appropriately used. Examples thereof include a communication device using wireless or wired communication.

Such a hardware configuration described above makes it possible to realize the processing function of the performance evaluation device 100 of a real-time PCR apparatus.

(Functional Configuration of Performance Evaluation Device)

FIG. 23 is a block diagram showing an example of a functional configuration of the performance evaluation device 100 of the real-time PCR apparatus. As shown in FIG. 23, the performance evaluation device 100 has an input unit 110, an output unit 120, a control unit 130, and a memory unit 140.

The control unit 130 has an information acquisition unit 131 and an evaluation unit 132. The control unit 130 controls the performance evaluation device 100 on the whole. The memory unit 140 has an information database 141 and an evaluation result database 142. Hereinafter, “database” may also be referred to as “DB”. The information acquisition unit 131 acquires information on the amplification reaction using the data stored in the information DB 141 of the memory unit 140. In the information DB 141, for example, data such as a Cq value obtained in advance by an experiment as described above is stored.

The information associated with the device may be stored in the information DB 141. The input to the DB may be carried out by other information-processing apparatuses connected to the performance evaluation device 100 or may be carried out by an operator.

The evaluation unit 132 evaluates the performance of a real-time PCR apparatus based on the information on the amplification reaction. The specific method of evaluating the performance of a real-time PCR apparatus is as described above. The performance evaluation result of a real-time PCR apparatus obtained by the evaluation unit 132 is stored in the evaluation result DB 142 of the memory unit 140.

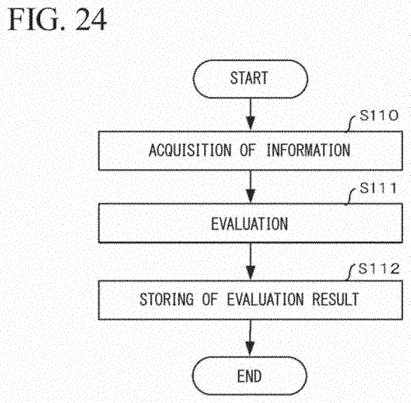

Subsequently, the processing procedure of the performance evaluation program of the present embodiment is described. FIG. 24 is a flowchart showing the processing procedure of the performance evaluation program in the control unit 130 of the performance evaluation device 100 of the real-time PCR apparatus.

In the step S110, the information acquisition unit 131 of the control unit 130 of the performance evaluation device 100 acquires the information data of the amplification reaction stored in the information DB 141 of the memory unit 140, and moves the process to the step S111.

In the step S111, the evaluation unit 132 of the control unit 130 of the performance evaluation device 100 evaluates the performance of a real-time PCR apparatus based on the acquired information and moves the process to the step S112.

In the step S112, the control unit 130 of the performance evaluation device 100 stores the obtained performance evaluation result of a real-time PCR apparatus in the evaluation result DB 142 of the memory unit 140 and ends the process.

### EXAMPLES

Next, the present invention will be described in more detail by showing Examples, but the present invention is not limited to Examples below.

### Experimental Example 1

(Manufacturing of Device)

A device in which a specific number of copies of a nucleic acid were immobilized on a reaction field (a bottom surface) of a well was manufactured by an inkjet method.

<<Preparation of Genetically Recombinant Yeast>>

Saccharomyces cerevisiae w303-1a (trade name: ATCC4001408, manufactured by ATCC) was used to prepare a recombinant, as a carrier cell of one copy of a specific DNA sequence. The specific DNA sequence, denoted by SEQ ID NO: 1, was made to tandemly align with URA3 as a selectable marker, and one copy of the specific DNA sequence was introduced into the yeast chromosome by homologous recombination in the BAR1 region of the carrier cell, whereby a genetically recombinant yeast was prepared.

<<Culture and Cell Cycle Control>>

Subsequently, 900 μL of the α factor (the α1-Mating Factor yeast salt, manufactured by Sigma-Aldrich Co., LLC) prepared to 500 μg/mL using Dulbecco's phosphate-buffered saline (manufactured by Thermo Fisher Scientific. Inc., hereinafter, may be referred to as “DPBS”) was added to an Erlenmeyer flask in which 90 mL of the genetically recombinant yeast cultured in a 50 g/L YPD medium (trade name: YPD Medium, manufactured by Clontech Laboratories, Inc.) was aliquoted, and the yeast was incubated using Bioshaker (device name: BR-23FH, manufactured by TIETECH Co., Ltd.) at a shaking rate of 250 rpm and a temperature of 28° C. for 2 hours and synchronized in the G0/G1 phase, whereby a yeast suspension was obtained. For confirming the cell cycle of synchronized cells, the cells were stained using SYTOX Green Nucleic Acid Chain (device name: S7020, manufactured by Thermo Fisher Scientific, Inc.), and the flow cytometry was carried out using a flow cytometer (device name: SH800, manufactured by Sony Corporation) at an excitation wavelength of 488 nm, whereby it was confirmed that the cells were synchronized in the G0/G1 phase. The G1 phase proportion was 97.1%, and the G2 phase proportion was 2.9%.

<<Fixing>>

Subsequently, 45 mL of the synchronization-confirmed yeast suspension was transferred to a centrifuge tube (Violamo, trade name: VIO-50R, manufactured by AS ONE Corporation), and centrifuged using a centrifuge (device name: CF16RN, manufactured by Hitachi, Ltd.) at a rotation speed of 3,000 rpm for 5 minutes, and the supernatant was removed to obtain a yeast pellet. 4 mL of formalin (manufactured by FUJIFILM Wako Pure Chemical Corporation, 062-01661) was added to the obtained yeast pellet, followed by allowing to stand for 5 minutes and then centrifuging to remove the supernatant, and 10 mL of ethanol was added to the pellet to suspend it, whereby a fixed yeast suspension was obtained.

<<Staining>>

Subsequently, 500 μL of the fixed yeast suspension was transferred to a 1.5 mL light-shielding tube (131-915BR, manufactured by WATSON Co., Ltd.) and centrifuged using a centrifuge at a rotation speed of 3,000 rpm for 5 minutes. The supernatant was removed, 400 μL of DPBS (1 mM EDTA) prepared to contain 1 mM EDTA (200-449-4, manufactured by TOCRIS Bioscience) was added to the yeast, followed by sufficiently suspending by pipetting. The suspension was centrifuged using a centrifuge at a rotation speed of 3,000 rpm for 5 minutes, and the supernatant was removed, whereby a yeast pellet was obtained. 1 mL of an Evans blue aqueous solution (054-04061, manufactured by FUJIFILM Wako Pure Chemical Corporation) prepared to 1 mg/mL was added to the obtained pellet, stirred for 5 minutes using a vortex, and then centrifuged using a centrifuge at a rotation speed of 3,000 rpm for 5 minutes. The supernatant was removed, and DPBS (1 mM EDTA) was added, and the mixture was stirred with a vortex, whereby a stained yeast suspension was obtained.

<<Dispersing>>

Subsequently, the stained yeast suspension was subjected to dispersion treatment using an ultrasonic homogenizer (device name: LUH150, Yamato Scientific Co., Ltd.) at a power output of 30% for 10 seconds and centrifuged using a centrifuge at a rotation speed of 3,000 rpm for 5 minutes. The supernatant was removed, and 1,000 μL of DPBS was added for washing. Centrifugation and removal of the supernatant were carried out twice in total, and finally, the yeast pellet was suspended in DPBS, whereby a yeast suspension ink was obtained.

<<Dispensing and Cell Counting>>

The number of yeasts in the liquid droplet was counted as follows to prepare a plate having a known number of cells. Specifically, using the liquid droplet-forming device shown in FIG. 8, the yeast suspension ink was sequentially ejected at 10 Hz using a piezoelectric application type ejection head (manufactured in-house) as a liquid droplet ejection means, into each well of a 96-well plate (trade name: MicroAmp 96-well Reaction plate, manufactured by Thermo Fisher Scientific. Inc.), the yeast in the ejected liquid droplet was imaged using a high-sensitivity camera (sCMOS pco edge, manufactured by Tokyo Instruments, Inc.) as a light-receiving means and a YAG laser (Explorer ONE-532-200-KE, manufactured by Spectra-Physics KK.) as a light source, and image processing was carried out to measure the number of cells using Image J, which is an image-processing software, as a particle number-measuring means for the taken image, whereby a plate having a known number of cells was prepared.

<<Nucleic Acid Extraction>>

ColE1/TE was prepared at 5 ng/μL using Tris-EDTA (TE) Buffer and ColE1 DNA (312-00434, manufactured by FUJIFILM Wako Pure Chemical Corporation), and a Zymolyase solution of Zymolyase (registered trademark) 100T (07665-55, manufactured by Nacalai Tesque, Inc.) was prepared at 1 mg/mL using the ColE1/TE. 4 μL of the Zymolyase solution was added to each well of the prepared plate having a known number of cells, incubated at 37° C. for 30 minutes to carry out digesting the cell wall (the nucleic acid extraction), and then treated at 95° C. for 2 minutes to prepare a plate.

Subsequently, the plate was dried by heating at 40° C. for 60 minutes, 60° C. for 30 minutes, or 80° C. for 15 minutes. As a control, a plate without heating and drying was prepared.

<<PCR Reaction>>

Next, the %-well plate was centrifuged, the number of cells was counted, primers, an enzyme, and water were added to the well having one cell, and a PCR reaction was carried out using a real-time PCR apparatus (product name “Quant Studio (trademark) 12K Flex Real-Time PCR System” (Applied Biosystems)). The proportion of the number of wells in which amplification occurred to the number of wells having one cell was evaluated as the detection rate.

As primers, a forward primer (SEQ ID NO: 2) and a reverse primer (SEQ ID NO: 3) were used. The concentration of the primer was 0.5 μM for the forward primer (SEQ ID NO: 2) and 0.5 μM for the reverse primer (SEQ ID NO: 3).

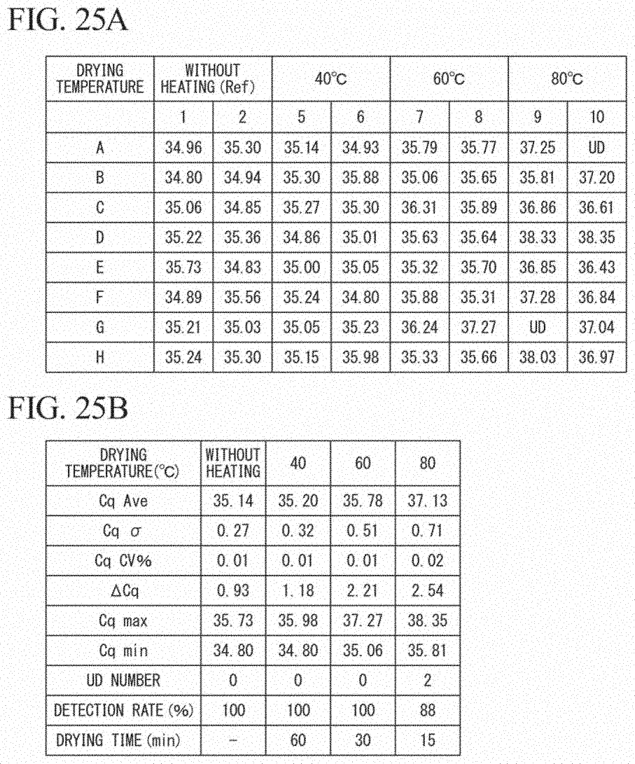

FIG. 25A is a table showing the in-plane distribution of Cq values of a targeted amplification product. FIG. 25A is a table showing evaluation results of the amplification reaction. In FIG. 25A, “A” to “H” in the leftmost column indicate row symbols of the 96-well plate, “1” to “10” in the top row indicate column numbers of the 96-well plate, and “UD” indicates a well in which amplification was not observed.

In addition, in FIG. 25B, “Cq Ave” indicates the average value of the Cq values, “Cq a” indicates the standard deviation of the Cq values, “Cq CV %” indicates the CV value of the Cq value, “ΔCq” indicates the in-plane difference of the Cq value (Cq max−Cq min), “Cq max” indicates the maximum value of the Cq value, “Cq min” indicates the minimum value of the Cq value, “UD number” indicates the number of wells in which amplification was not observed, and “Detection rate” indicates a value calculated by the following Expression 6.

Detection rate (%)=number of wells in which nucleic acid amplification was detected/(number of wells subjected to amplification reaction−number of wells of negative control)×100 Expression 6

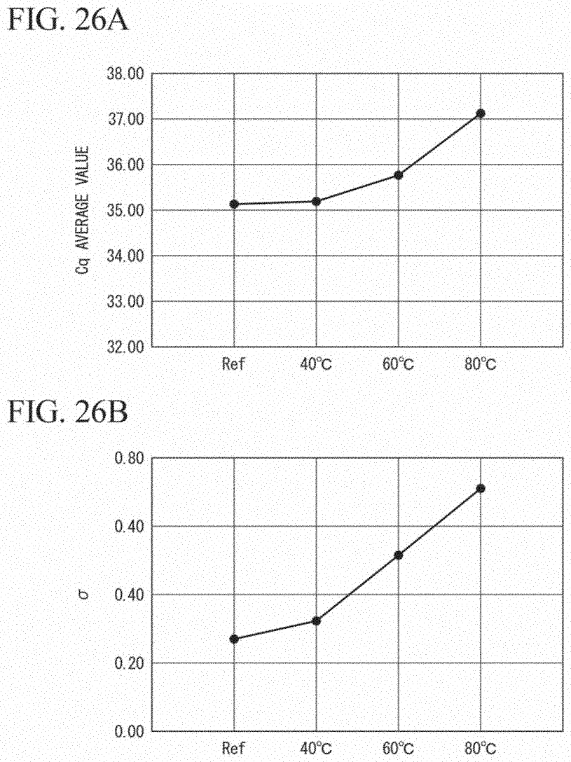

FIG. 26A is a graph showing the relationship between the heating temperature and Cq Ave. The vertical axis of the graph shows Cq Ave, and the horizontal axis of the graph shows the heating temperature. FIG. 26B is a graph showing the relationship between the heating temperature and Cq a. The vertical axis of the graph shows Cq a, and the horizontal axis of the graph shows the heating temperature.

From the result, it was found that both Cq Ave and Cq σ increase as the drying temperature of the plate increases. From this, it was revealed that a PCR reaction cannot be accurately evaluated in a case where the plate is heated and dried.

### Experimental Example 21

The plate in which nucleic acid was extracted in the same manner as in Experimental Example 1 was evacuated at room temperature (23° C.) in a vacuum of about 1 Mpa for 3 hours using a vacuum dryer to dry the liquid in the container.

Subsequently, a PCR reaction was carried out in the same manner as in Experimental Example 1, and Cq Ave and Cq σ were calculated. The results are shown in Table 2. In Table 2, “A” to “H” in the leftmost column indicate the row symbols of the 96-well plate. “Without drying” indicates Cq values of the plate that was not vacuum-dried, and “With drying” indicates Cq values of the plate that was vacuum-dried.

TABLE 2

| | Cq value | |
|---|---|---|
| Column | Without drying | With drying |
| A | 34.83 | 35.21 |
| B | 34.96 | 35.19 |
| C | 35.08 | 35.09 |
| D | 35.34 | 35.42 |
| E | 34.59 | 35.28 |
| F | 34.98 | 34.66 |
| G | 34.83 | 35.16 |
| H | 35.23 | 34.91 |
| Cq Ave | 34.98 | 35.12 |
| Cq σ | 0.24 | 0.23 |

As shown in Table 2, none of Cq Ave and Cq σ changed significantly depending on whether the plate was dried or not.

### Experimental Example 3

After digesting the cell wall (the nucleic acid extraction), nucleic acid extraction was carried out in the same manner as in Experimental Example 1 to prepare a plate except that deactivation of Zymolyase by treatment at 95° C. for 2 minutes was not carried out. Then, using a vacuum dryer, the liquid in the container was dried by vacuuming at room temperature (23° C.) in a vacuum of about 1 Mpa for 3 hours. Zymolyase is deactivated by drying under reduced pressure.

Subsequently, the obtained plate was stored at 40° C. for 6 days, and a PCR reaction was carried out in the same manner as in Experimental Example 1 to calculate Cq Ave and Cq σ.

The plate obtained in Experimental Example 2 was also stored at 40° C. for 6 days, and a PCR reaction was carried out in the same manner as in Experimental Example 1 to calculate Cq Ave and Cq a. The results are shown in Table 3 below. In Table 3, “A” to “H” in the leftmost column indicate the row symbols of the 96-well plate, “Deactivation by heating” indicates the Cq value of the plate of Experimental Example 2, where Zymolyase was treated at 95° C. for 2 minutes to deactivate Zymolyase and dried under reduced pressure, “Deactivation by drying” indicates the Cq value of the plate of Experimental Example 3, where Zymolyase was deactivated by drying under reduced pressure without treating Zymolyase at 95° C. for 2 minutes.

TABLE 3

| | Cq value | |
|---|---|---|
| | Deactivation by | Deactivation by |
| Column | heating | drying |
| A | 35.61 | 35.03 |
| B | 36.30 | 35.05 |
| C | 35.66 | 35.12 |
| D | 35.08 | 35.21 |
| E | 36.08 | 35.24 |
| F | 35.23 | 34.95 |
| Ci | 36.76 | 35.92 |
| H | 34.81 | 35.19 |
| Cq Ave | 35.69 | 35.09 |
| Cq σ | 0.66 | 0.12 |

As shown in Table 3, it was revealed that in the plate in which Zymolyase was not subjected to deactivation by treatment at 95° C. for 2 minutes but deactivated by drying under reduced pressure at room temperature in the nucleic acid extraction, the changes in both Cq Ave and Cq σ were small and storage stability was excellent even in a case of being stored at 40° C. for 6 days as compared with the plate in which Zymolyase was subjected to deactivation by treatment at 95° C. for 2 minutes and dried under reduced pressure at room temperature.

### Experimental Example 4

A plate was prepared in the same manner as in Experimental Example 3. The obtained plate was vacuum-packed as it was or together with silica gel, stored at 23° C. for 28 days and then subjected to a PCR reaction in the same manner as in Experimental Example 1, and Cq Ave, Cq a, Cq CV %, ΔCq, Cq max, Cq min, the UD number, and detection rate were calculated. The results are shown in Table 4. In Table 4, “Room temperature” indicates that the plate was stored as it was at 23° C., “Room temperature vacuum” indicates that the plate vacuum-packed together with silica gel was stored at 23° C., and “Initial value” indicates Cq σ before storage.

TABLE 4

| | | Room temperature |
|---|---|---|
| Storage condition | Room temperature | vacuum |
| Cq Ave | 35.61 | 35.49 |
| Cq σ | 0.40 | 0.34 |
| Cq CV % | 0.011 | 0.010 |
| ΔCq | 1.88 | 1.94 |
| Cq max | 36.65 | 36.65 |
| Cq min | 34.78 | 34.71 |
| UD number | 0 | 0 |
| Detection rate | 100% | 100% |
| Initial value | 0.42 | 0.30 |

As shown in Table 4, it was revealed that in the plate in which Zymolyase was not subjected to deactivation by treatment by 95° C. for 2 minutes but deactivated by drying under reduced pressure at room temperature in the nucleic acid extraction, storage stability was excellent even in a case of being stored at 23° C. for 28 days due to being vacuum-packed together with silica gel.

### Experimental Example 5

Using an 8-well strip tube instead of the 96-well plate, the cell suspension was ejected into an 8-well strip tube cap (MicroAmp (trademark) Optical 8-Cap Strip, manufactured by Thermo Fisher Scientific, Inc.), and after digesting the cell wall (the nucleic acid extraction), an 8-well strip tube cap on which the nucleic acid was immobilized was prepared in the same manner as in Experimental Example 1 except that deactivation of Zymolyase by treatment at 95° C. for 2 minutes was not carried out. Then, using a vacuum dryer, the liquid in the cap was dried by vacuuming at room temperature (23° C.) in a vacuum of about 1 Mpa for 3 hours.

Next, an 8-well strip tube (MicroAmp (trademark) Fast Reaction Tubes, manufactured by Thermo Fisher Scientific, Inc.) containing primers, an enzyme, and water (hereinafter, referred to as a PCR reaction solution) was sealed with the 8-well strip tube cap obtained above, the tube sealed with the cap was turned upside down and allowed to stand for 2 minutes, and then mixed by inverting for 10 times, whereby an insoluble carrier sample was obtained.

Subsequently, the insoluble carrier sample obtained above was centrifuged, and a PCR reaction was carried out using a real-time PCR apparatus (product name “Quant Studio™ 12K Flex Real-Time PCR System” (Applied Biosystems)).

Next, an 8-well strip tube was used instead of the 96-well plate, and after digesting the cell wall (the nucleic acid extraction), an 8-well strip tube having the nucleic acid immobilized on the bottom thereof was prepared in the same manner as in Experimental Example 1 except that deactivation of Zymolyase by treatment at 95° C. for 2 minutes was not carried out. Then, using a vacuum dryer, the liquid in the tube was dried by vacuuming at room temperature (23° C.) in a vacuum of about 1 Mpa for 3 hours, whereby a control sample (a reference) was prepared.

A PCR reaction solution was added to the control sample, centrifuged, and a PCR reaction was carried out using a real-time PCR apparatus (product name “Quant Studio™ 12K Flex Real-Time PCR System” (Applied Biosystems)).

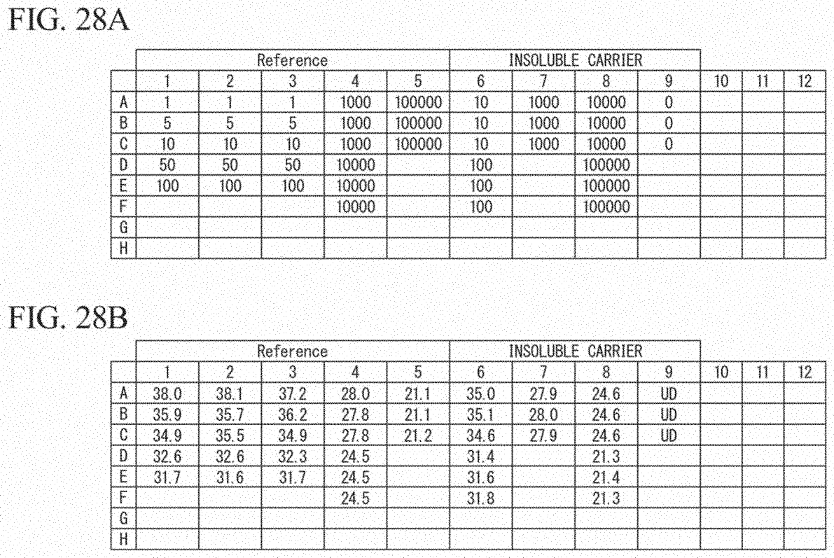

FIG. 28A is a table showing theoretical copy numbers of the amplification products of the control sample and the insoluble carrier sample. FIG. 28B is a table showing the in-plane distribution of Cq values of the amplification products of the control sample and the insoluble carrier sample. In FIG. 28A and FIG. 28B, “A” to “H” in the leftmost column indicate the position of each of the tubes of the 8-well strip tube, and “1” to “9” in the top row indicate each of the 8-well strip tubes (tubes No. 1 to No. 9). The blank indicates an empty tube in which no sample was added, and “UD” indicates a tube in which amplification was not observed. The theoretical copy number refers to the copy number of DNA, calculated from the measured number of cells in the liquid droplet.

Next, Cq Ave, Cq a, Cq CV %, ΔCq, Cq max, and Cq min of the control sample and the insoluble carrier sample were calculated. The results of the control sample are shown in Table 5, and the results of the insoluble carrier sample are shown in Table 6. In the insoluble carrier sample, the Cq Ave difference from the Cq Ave of the control sample was also calculated.

TABLE 5

| Theoretical | | | | | |
|---|---|---|---|---|---|
| copy number | 10 | 100 | 1000 | 10000 | 100000 |
| Cq AVC | 35.11 | 31.68 | 27.84 | 24.48 | 21.10 |
| Cq σ | 0.37 | 0.05 | 0.11 | 0.03 | 0.06 |
| Cq CV % | 1.06 | 0.17 | 0.41 | 0.13 | 0.30 |
| ΔCq | 0.68 | 0.10 | 0.21 | 0.06 | 0.11 |
| Cq max | 35.54 | 31.73 | 27.97 | 24.52 | 21.17 |
| Cq min | 34.85 | 31.63 | 27.76 | 24.46 | 21.06 |

TABLE 6

| Theoretical | | | | | |
|---|---|---|---|---|---|
| copy number | 10 | 100 | 1000 | 10000 | 100000 |
| Cq Ave | 34.93 | 31.60 | 27.94 | 24.61 | 21.32 |
| Cq σ | 0.28 | 0.20 | 0.04 | 0.03 | 0.06 |
| Cq CV % | 0.79 | 0.62 | 0.13 | 0.11 | 0.30 |
| ΔCq | 0.53 | 0.39 | 0.07 | 0.05 | 0.12 |
| Cq max | 35.15 | 31.81 | 27.97 | 24.64 | 21.37 |
| Cq min | 34.62 | 31.41 | 27.90 | 24.59 | 21.25 |
| Cq difference | −0.18 | −0.07 | 0.10 | 0.13 | 0.22 |
| from control | | | | | |

As shown in Table 5 and Table 6, the Cq values of the insoluble carrier sample and the control sample were almost the same. From this, it was revealed that even in a case where a nucleic acid is immobilized on the cap of the 8-well strip tube, the nucleic acid is eluted from the cap without being degraded and is amplified by the PCR reaction when the nucleic acid immobilized on the cap is dried under reduced pressure.

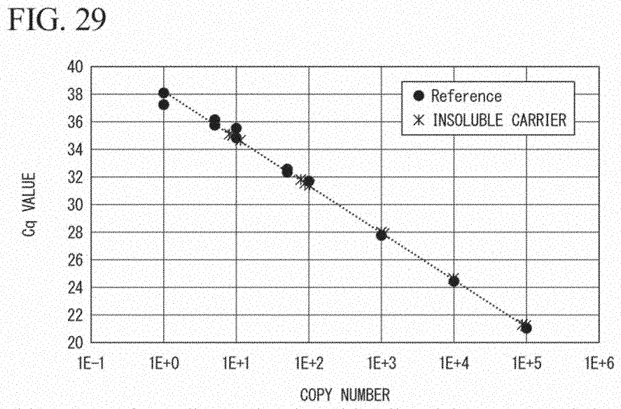

Next, a calibration curve was created from the theoretical copy number and the Cq value of the control sample. The created calibration curve is shown in FIG. 29. From this calibration curve, it was found that the relationship between the Cq value and the copy number is expressed by the following Expression 7.

Copy number=10\hat{ }((Cq value−38.218)/−3.413) Expression 7

Using Expression 7, the copy numbers were calculated from the Cq values of the control sample and the insoluble carrier sample. The results are shown in Table 7.

TABLE 7

| | Reference | | | | | Insoluble carrier | | | | | | |
|---|---|---|---|---|---|---|---|---|---|---|---|---|
| | 1 | 2 | 3 | 4 | 5 | 6 | 7 | 8 | 9 | 10 | 11 | 12 |
| A | 1 | 1 | 2 | 1006 | 106703 | 9 | 1031 | 9525 | 0 | | | |
| B | 5 | 6 | 4 | 1158 | 106234 | 8 | 1004 | 9865 | 0 | | | |
| C | 9 | 6 | 10 | 1137 | 98924 | 11 | 1054 | 9807 | 0 | | | |
| D | 46 | 44 | 54 | 10793 | | 99 | | 93821 | | | | |
| E | 83 | 86 | 80 | 10639 | | 87 | | 86515 | | | | |
| F | | | | 10360 | | 76 | | 87990 | | | | |
| G | | | | | | | | | | | | |
| H | | | | | | | | | | | | |

From the results in Table 7, the copy numbers of the control sample and the insoluble carrier sample were evaluated. The evaluation results of the copy number of the control sample are shown in Table 8, and the evaluation results of the copy number of the insoluble carrier sample are shown in Table 9. In Table 8 and Table 9, “Ave” indicates the average copy number of 3 tubes, “a” indicates the SD copy number of 3 tubes, “CV %” indicates (σ/Ave)×10, “max” indicates the maximum copy number of 3 tubes, “min” indicates the minimum copy number of 3 tubes, “max−min” indicates the value obtained by subtracting the minimum value from the maximum value, and “Elution rate” indicates a value calculated by the following Expression 8.

Elution rate (%)=Ave of insoluble carrier sample/theoretical copy number Expression 8

TABLE 8

| Theoretical | | | | | |
|---|---|---|---|---|---|
| copy number | 10 | 100 | 1000 | 10000 | 100000 |
| Ave | 8 | 83 | 1100 | 10597 | 103953 |
| σ | 2 | 3 | 82 | 220 | 4362 |
| CV % | 23.2 | 3.5 | 7.4 | 2.1 | 4.2 |
| max - min | 3.6 | 5.8 | 151.1 | 433.2 | 7778.9 |
| max | 10 | 86 | 1158 | 10793 | 106703 |
| min | 6 | 80 | 1006 | 10360 | 98924 |

TABLE 9

| Theoretical | | | | | |
|---|---|---|---|---|---|
| copy number | 10 | 100 | 1000 | 10000 | 100000 |
| Ave | 9 | 87 | 1030 | 9732 | 89442 |
| σ | 1.8 | 11.5 | 25.3 | 182.1 | 3863.1 |
| CV % | 19.3 | 13.2 | 2.5 | 1.9 | 4.3 |
| max - min | 3.4 | 23.1 | 50.5 | 340.5 | 7305.6 |
| max | 11 | 99 | 1054 | 9865 | 93821 |
| min | 8 | 76 | 1004 | 9525 | 86515 |
| Elution rate (%) | 92.9 | 87.2 | 103.0 | 97.3 | 89.4 |

As shown in Table 8 and Table 9, regarding the copy number of DNA calculated from the calibration curve as well, the copy number of DNA of the insoluble carrier sample is equivalent to the copy number of DNA of the control sample, and the elution rate from the cap is as high as 89.4% or more. From this as well, it was revealed that even in a case where a nucleic acid is immobilized on the cap of the 8-well strip tube, the nucleic acid is eluted from the cap without being degraded and is amplified by the PCR reaction when the nucleic acid immobilized on the cap is dried under reduced pressure.

The present invention includes the following aspects.

[1] A method for manufacturing a device having at least one well, in which a specific number of copies of a nucleic acid in the at least one well are immobilized in a reaction field, the method including a nucleic acid extraction step of extracting the nucleic acid with an enzyme and a drying deactivation step of deactivating the enzyme by drying at 5° C. to 45° C.

[2] The method for manufacturing a device according to [1], in which the immobilization of the nucleic acid to the reaction field is immobilization to a bottom surface of the well.

[3] The method for manufacturing a device according to [1], in which the immobilization of the nucleic acid to the reaction field is immobilization to an insoluble carrier that comes into contact with the reaction field.

[4] The method for manufacturing a device according to [1], in which the immobilization of the nucleic acid to the reaction field is addition of the nucleic acid immobilized to an insoluble carrier to the reaction field.

[5] The method for manufacturing a device according to any one of [1] to [4], in which the drying is drying under reduced pressure.

[6] The method for manufacturing a device according to any one of [1] to [5], in which the nucleic acid is incorporated into a nucleic acid in a nucleus of a cell.

[7] The method for manufacturing a device according to any one of [1] to [6], in which the specific number of copies is 1 copy or more and 200 copies or less.

[8] A device including at least one well, in which a specific number of copies of a nucleic acid in the at least one well are immobilized in a reaction field, and the nucleic acid is extracted with an enzyme and immobilized in the reaction field by drying the enzyme at 5° C. to 45° C.

[9] The device according to [8], in which the immobilization of the nucleic acid to the reaction field is immobilization to a bottom surface of the well.

[10] The device according to [8], in which the immobilization of the nucleic acid to the reaction field is immobilization to an insoluble carrier that comes into contact with the reaction field.

[11] The device according to [8], in which the immobilization of the nucleic acid to the reaction field is addition of the nucleic acid immobilized to an insoluble carrier to the reaction field.

[12] The device according to any one of [8] to [11], in which the specific number of copies is 1 copy or more and 200 copies or less.

[13] A performance evaluation kit for a real-time PCR apparatus, including the device according to any one of [8] to [12].

While preferred embodiments of the invention have been described and illustrated above, it should be understood that these are exemplary of the invention and are not to be considered as limiting. Additions, omissions, substitutions, and other modifications can be made without departing from the scope of the invention. Accordingly, the invention is not to be considered as being limited by the foregoing description and is only limited by the scope of the appended claims.

Sequences

The present application comprises
the following sequences:
SEQ ID NO: 1 (Synthesized oligonucleotide)
attcgaaggg tgattggatc ggagatagga tgggtcaatc
gtagggacaa tcgaagccag aatgcaaggg tcaatggtac
gcagaatgga tggcacttag ctagccagtt aggatccgac
tatccaagcg tgtatcgtac ggtgtatgct tcggagtaac
gatcgcacta agcatggctc aatcctaggc tgataggttc
gcacatagca tgccacatac gatccgtgat tgctagcgtg
attcgtaccg agaactcacg ccttatgact gcccttatgt
caccgcttat gtctcccgag atcacacccg ttatctcagc
cctaatctct gcggtttagt ctggccttaa tccatgcctc
atagctaccc tcataccatc gctcatacct tccgacattg
catccgtcat tccaaccctg attcctacgg tctaacctag
cctctatcct acccagttag gttgcctctt agcatccctg
ttacgtacgc tcttaccatg cgtcttacct tggcactatc
gatgggagta tggtagcgag tatggaacgg actaacgtag
gcagtaagct agggtgtaag gttgggacta aggatgccag
SEQ ID NO: 2 (Synthesized oligonucleotide)
tcgaagggtg attggatcgg
SEQ ID NO: 3 (Synthesized oligonucleotide)
tggctagcta agtgccatcc

### EXPLANATION OF REFERENCES

1 . . . Device
2 . . . Base material
3 . . . Reaction space (well)
4 . . . Nucleic acid
5 . . . Sealing member
6 . . . Identification means
10, 10′, 10C . . . Ejection head (liquid droplet-ejecting means)
11, 11a, 11b, 11c, 11C, 11′ . . . Liquid chamber
12, 12C . . . Membrane
13, 13C . . . Driving element
13a . . . Electric motor
13b, 13c . . . Piezoelectric element
20 . . . Driving means
30, 260 . . . Light source
40 . . . Mirror
60, 61 . . . Light-receiving element
70 . . . Controlling means
71, 101 . . . CPU
72 . . . ROM
73 . . . RAM
74, 106 . . . I/F
75 . . . Bus line
100 . . . Performance evaluation device
102 . . . Main memory device
103 . . . Auxiliary memory device
104 . . . Output device
105 . . . Input device
107 . . . Bus
111, 111a, 111b, 111c, 121 . . . Nozzle
112 . . . Solenoid valve
115 . . . Atmospheric air opening part
200 . . . Coil
250 . . . Micro flow path
255 . . . Detector
255′ . . . Image acquisition part
265,265′ . . . Lens
300, 300a, 300b, 300c . . . Cell suspension
310, 310′ . . . Liquid droplet
350, 350₁, 350₂, 350′, 350″ . . . Cell
400 . . . Dispensing device
401, 401A, 401B, 401C . . . Liquid droplet-forming device
700, 700′ . . . Plate
710 . . . Well
800 . . . Stage
900 . . . Control device
L . . . Light
Lf, Lf₁, Lf₂ . . . Fluorescence

[Patent Document 1] Japanese Unexamined Patent Application, First Publication No. 2019-216703

What is claimed is:

1. A method for manufacturing a device having at least one well, in which a specific number of copies of a nucleic acid in the at least one well are immobilized in a reaction field, the method comprising:
a nucleic acid extraction step of extracting the nucleic acid with an enzyme; and
a drying deactivation step of deactivating the enzyme by drying at 5° C. to 45° C.

2. The method for a manufacturing device according to claim 1,
wherein the immobilization of the nucleic acid to the reaction field is immobilization to a bottom surface of the well.

3. The method for manufacturing a device according to claim 1,
wherein the immobilization of the nucleic acid to the reaction field is immobilization to an insoluble carrier that comes into contact with the reaction field.

4. The method for manufacturing a device according to claim 1,
wherein the immobilization of the nucleic acid to the reaction field is addition of the nucleic acid immobilized to an insoluble carrier to the reaction field.

5. The method for manufacturing a device according to claim 1,
wherein the drying is drying under reduced pressure.

6. The method for manufacturing a device according to claim 1,
wherein the nucleic acid is incorporated into a nucleic acid in a nucleus of a cell.

7. The method for manufacturing a device according to claim 1,
wherein the specific number of copies is 1 copy or more and 200 copies or less.

8. A device comprising:
at least one well,
wherein a specific number of copies of a nucleic acid in the at least one well are immobilized in a reaction field, and
the nucleic acid is extracted with an enzyme and immobilized in the reaction field by drying the enzyme at 5° C. to 45° C.

9. The device according to claim 8,
wherein the immobilization of the nucleic acid to the reaction field is immobilization to a bottom surface of the well.

10. The device according to claim 8,
wherein the immobilization of the nucleic acid to the reaction field is immobilization to an insoluble carrier that comes into contact with the reaction field.

11. The device according to claim 8,
wherein the immobilization of the nucleic acid to the reaction field is addition of the nucleic acid immobilized to an insoluble carrier to the reaction field.

12. The device according to claim 8,
wherein the specific number of copies is 1 copy or more and 200 copies or less.

13. A performance evaluation kit for a real-time PCR apparatus, comprising the device according to claim 8.


SEQUENCE LISTING (3 sequences)

SEQ ID NO: 1 - DNA, 600 bp, Artificial Sequence
  Synthesized oligonucleotide
attcgaagggtgattggatcggagataggatgggtcaatcgtagggacaatcgaagccagaatgcaagggtcaatggtacgcagaatggatggcacttagctagccagttaggatccgactatccaagcgtgtatcgtacggtgtatgcttcggagtaacgatcgcactaagcatggctcaatcctaggctgataggttcgcacatagcatgccacatacgatccgtgattgctagcgtgattcgtaccgagaactcacgccttatgactgcccttatgtcaccgcttatgtctcccgagatcacacccgttatctcagccctaatctctgcggtttagtctggccttaatccatgcctcatagctaccctcataccatcgctcataccttccgacattgcatccgtcattccaaccctgattcctacggtctaacctagcctctatcctacccagttaggttgcctcttagcatccctgttacgtacgctcttaccatgcgtcttaccttggcactatcgatgggagtatggtagcgagtatggaacggactaacgtaggcagtaagctagggtgtaaggttgggactaaggatgccag

SEQ ID NO: 2 - DNA, 20 bp, Artificial Sequence
  Synthesized oligonucleotide
tcgaagggtgattggatcgg

SEQ ID NO: 3 - DNA, 20 bp, Artificial Sequence
  Synthesized oligonucleotide
tggctagctaagtgccatcc